(19) United States
(12) Patent Application Publication
(10) Pub. No.: US 2003/0166725 A1
(43) Pub. Date: Sep. 4, 2003

(54) Compounds active at the glucocorticoid receptor

(76) Inventors: Theresa Apelgvist, Huddinge (SE); Mikael Gillner, Stockholm (SE); Annika Gustavsson, Ekero (SE); Lars Hagberg, Stockholm (SE); Eva Koch, Stockholm (SE); Marita Nilsson, Huddinge (SE); Benjamin Pelcman, Stockholm (SE); Jinchang Wu, Huddinge (SE); Philip R. Kym, Grayslake (SE)

Correspondence Address: Wiggin & Dana Docket Coordinator, Intellectual Property Law Section, One Century Tower, New Haven, CT, 06508-1832, US

(21) Appl. No.: 10/148,721

(22) PCT Filed: Dec. 6, 2000
(86) PCT No.: PCT/IB00/01927

(30) Foreign Application Priority Data
Dec. 7, 1999 (GB) 9928805.2

(51) Int. Cl. A61K 31/195; A61K 31/192

(52) U.S. Cl. 514/567; 514/570; 562/465; 562/451

(57) ABSTRACT

This invention relates to novel compounds that are liver selective glucocorticoid receptor antagonists, to methods of preparing such compounds, and to methods for using such compounds in therapy and in the regulation of metabolism, especially lowering blood glucose levels. The compounds referred to are compounds according to the formula (I).

### FIELD OF THE INVENTION

This invention relates to novel compounds that are liver selective glucocorticoid receptor antagonists, to methods of preparing such compounds, and to methods for using such compounds in therapy and in the regulation of metabolism, especially lowering blood glucose levels.

### BACKGROUND OF THE INVENTION

A major problem with both Type 2 and Type 1 diabetes is that there is an excessive and inappropriate production of glucose by the liver. This abnormality is the primary cause of fasting hyperglycemia and occurs in addition to defects in regulation of insulin release and in peripheral sensitivity to insulin. Thus, agents that decrease liver glucose production would be beneficial for treating both Type 2 and also Type 1 diabetes. Intensive treatment of the hyperglycemia of Type 1 diabetes mellitus has been shown to markedly decrease the development of ocular, renal and neuropathic complications, and there is evidence that intensive treatment is also beneficial for Type 2 diabetes. The available data also indicate that most patients are currently not receiving ideal and state-of-the-art treatment for either Type 2 or Type 1 diabetes. This inadequacy exists in spite of the availability of several different types of preparations of insulin for treatment of both Type 2 and Type 1 diabetes, and of a number of additional modalities, including agents that stimulate insulin release (e.g. sulfonylureas), influence liver glucose production (e.g. mefformin), affect the sensitivity to insulin (e.g. compounds interacting with the PPARγ such as troglitazone, rosiglitazone and pioglitazone) and glucose absorption (e.g. α-glucosidase inhibitors such as acarbose). In spite of the availability of several different orally active agents that lower blood glucose levels, many patients with Type 2 diabetes also require insulin for control of their blood sugar levels. Overall, insulin usage in Type 2 diabetes exceeds that for Type 1 diabetes, and there is general agreement that there is a need for additional orally active agents to treat Type 2 diabetes.

The glucocorticoids secreted from the adrenal gland (dominantly cortisol in humans) were so-named because of their ability to regulate glucose metabolism. These steroids stimulate the production of glucose in the liver by promoting gluconeogenesis, which is the biosynthesis of new glucose (i.e. not glucose from glycogen). Thus, in glucocorticoid insufficiency there is a tendency to hypoglycemia, with decreased liver glucose production. Further, development of Addison's disease in the diabetic generally leads to lowered glucose levels. Conversely, glucocorticoid excess can provoke frank diabetes in individuals with latent diabetes mellitus, and generally aggravates glycemic control in established diabetics. Similar influences have been observed in various animal models.

The glucocorticoid receptor (GR) belongs to a large group of ligand dependent intracellular receptors, which regulate transcription of genes. The increased glucose production in response to glucocorticoids is due to effects of a number of proteins, which are GR regulated. Important among these proteins are various transaminases that convert amino acids to glucose precursors, glucose-6 phosphatase and phosphoenolpyruvate carboxy-kinase (PEPCK). Even a modest increase of PEPCK, as obtained in transgenic mice, gives rise to hyperglycemia. In mice with Type 2 diabetes and increased levels of corticosterone (the endogenous glucocorticoid of that species) there is increased expression of PEPCK. This over-expression of PEPCK can be repressed by treatment with the GR antagonist RU486 with a concomitant decrease in the hyperglycemia.

The considerations outlined above indicate that if actions of endogenous glucocorticoids on liver glucose production could be blocked in a specific manner, glycemic control could be improved for the benefit of the diabetic patients. However, to date, all means to block glucocorticoid action have been general. Thus, adrenalectomy leaves the patient with frank adrenal insufficiency and the problems of Addison's disease. Blockade of adrenal steroid production, for example by metyrapone, or of glucocorticoid action, for example with RU486 is ordinarily of limited duration of effectiveness and when it is effective also results in generalized adrenal insufficiency. Longterm, compensatory ACTH hypersecrefion and increased cortisol release that override the block generally overcome these treatments. By contrast, a liver-selective GR antagonist would not have these problems, but should yet counteract the increased liver glucose production in diabetes mellitus and should be useful for treatment of Type 2 diabetes.

A liver selective GR antagonist offers a number of advantages. First, it would decrease liver glucose production. This action will have a significant effect on glycemic control. In fact, excessive liver glucose production can be the major defect in Type 2 diabetes. Secondly, such a drug should enhance insulin sensitivity because of the overall improvement in the metabolic milieu and the amelioration of the hyperglycemia-induced defects in insulin action and secretion. The decreased demand on β-cell secretion, as a result of a reduction in glycemia, would retard the progressive β-cell dysfunction characteristic of Type 2 diabetes. Another advantage of GR antagonist treatment compared with sulfonylurea or insulin treatment is that the patient would run a lower risk of hypoglycemia.

Previous efforts to block glucocorticoid action in diabetes have been hampered by the fact that any compounds used would generally block glucocorticoid action in all tissues and would lead to the potential problems of glucocorticoid insufficiency, such as hypotension, shock and ultimately death if the organism is exposed to sufficiently strong stress conditions. In contrast, a liver-selective GR-antagonist with minimal effects outside the liver could be used as a front line therapy for Type 2 diabetes, or could be used in conjunction with other existing therapies.

Also, glucocorticoids are known to influence the development and/or maintenance of inflammation, autoimmune disease, transplant rejection, neoplasm, leukemia, lymphoma, Cushings disease, adrenal disease, renal disease, cerebrovascular ischemia, hypercalcemia, cerebral edema, thrombocytopenia, inflammatory bowel disease, wound healing, HIV infection, central nervous system disease, spinal cord tumour, glaucoma, sleep disorder, depression, anxiety disorder, atherosclerosis, hypertension, osteoporosis, occular hypertension, nephrotoxicity, infarction, endometriosis, pregnancy disorder, psychosis, Alzheimers disease, cocaine use disorder, asthma, allergic rhinitis, conjuctivitis, rheumatoid arthritis, dermatitis, eczema, osteoarthritis, hypoglycemia, hyperinsulinemia, hyperlipidemia and obesity.

### DESCRIPTION OF THE INVENTION

In accordance with the present invention, compounds are provided which are active at the glucocorticoid hormone receptor, and have the general formula I:

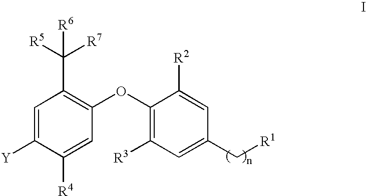

wherein:

R¹ is selected from:

COOH, C(O)NHOH, C(O)COOH, SO₃H, P(O)(OH)(OR⁸), P(O)(OH)[N(R⁹)(R¹⁰)], and heteroaryl, wherein any heteroaryl residue may be optionally substituted in one or more positions independently of each other by a group selected from C₁₋₆-alkyl, perfluoro-C₁₋₆-alkyl, halogen, cyano, nitro, R⁸O, R⁸S, R⁸S(O), R⁸S(O)₂ and (R⁹)(R¹⁰)N;

R² and R³ are independently of each other selected from:

hydrogen, halogen, C₁₋₆-alkyl, hydroxy, C₁₋₆-alkoxy, C₁₋₆-alkylthio, halo-C₁₋₆-alkyl, perfluoro-C₁₋₆-alkyl, halo-C₁₋₆-alkyloxy, perfluoro-C₁₋₆-alkyloxy, and halo-C₁₋₆-alkylthio, provided that one of R² or R³ is other than hydrogen;

R⁴, R⁵, R⁶ and R⁷ are independently of each other selected from:

(i) C₁₋₁₂-alkyl and perfluoro-C₁₋₆-alkyl, wherein any residues herein may be optionally substituted in one or more positions independently of each other by a group selected from A;

(ii) C₃₋₈-cycloalkyl, C₂₋₆-alkenyl, and C₂₋₆-alkynyl, wherein any residues herein may be optionally substituted in one or more positions independently of each other by a group selected from B;

R⁴ and R⁵ are optionally, and independently of each other, selected from:

(iii) C₃₋₈-heterocycloalkyl, optionally substituted by a group selected from B;

(iv) aryl and heteroaryl, wherein any residues herein may be optionally substituted in one or more positions independently of each other by a group selected from C;

R⁴ is optionally selected from:

halogen, R⁸O, R⁸S, R⁸S(O), R⁸S(O)₂, (R⁹)(R¹⁰)N, R⁸C(Z)N(R¹¹), (R⁹)(R¹⁰)NC(Z)N(R¹¹), R⁸S(O)₂N(R¹¹), and (R⁹)(R¹⁰)NS(O)₂N(R¹¹);

R⁶ and R⁷ are optionally, and independently of each other, selected from:

hydrogen, halogen, R⁸O, R⁸S, R⁸S(O), R⁸S(O)₂, (R⁹)(R¹⁰)N, R⁸C(Z)O, R⁸OC(Z)O, R⁸C(Z)N(R¹¹), R⁸OC(Z)N(R¹¹), R⁸S(O)ₙO, (R⁹)(R¹⁰)NC(Z)O, (R⁹)(R¹⁰)NS(O)₂O, R⁸S(O)₂N(R¹¹), (R⁹)(R¹⁰)NS(O)₂ and (R⁹)(R¹⁰)NS(O)₂N(R¹¹), provided that R⁸ is not hydrogen in R⁸OC(Z)O, R⁸S(O)ₙO, and R⁸S(O)₂N(R¹¹), and that only one of R⁶ and R⁷ is hydrogen, and that if R⁶ is HO, R⁷ is hydrogen, and that if R⁷ is HO, R⁶ is hydrogen;

R⁸, R⁹, R¹⁰ and R¹¹ are independently of each other selected from:

(v) hydrogen,

(vi) C₁₋₁₂-alkyl and perfluoro-C₁₋₆-alkyl, wherein any residues herein may be optionally substituted in one or more positions independently of each other by a group selected from A;

(vii) C₃₋₈-cycloalkyl, C₂₋₆-alkenyl, C₂₋₆-alkynyl, and C₃₋₈-heterocycloalkyl, wherein any residues herein may be optionally substituted in one or more positions independently of each other by a group selected from B;

(viii) aryl and heteroaryl, wherein any residues herein may be optionally substituted in one or more positions independently of each other by a group selected from C;

or where any pair of R⁸, R⁹, R¹⁰ and R¹¹ together with the atom or atoms to which they are bound form a ring having 3-7 ring members, and which ring optionally contain 1-3 heteroatoms, or 1-3 double bonds, and which optionally is substituted by a group selected from B;

A is selected from:

halogen, perfluoro-C₁₋₆-alkyl, C₃₋₈-cycloalkyl, C₂₋₆alkenyl, C₂₋₆-alkynyl, aryl, C₃₋₈-heterocycloalkyl, heteroaryl, cyano, nitro, azido, Z, R⁸O, R⁸C(Z), R⁸C(Z)O, R⁸OC(Z), R⁸S, R⁸S(O), R⁸S(O)₂, R⁸S(O)₂O, R⁸OS(O)₂, (R⁹)(R¹⁰)N, (R⁹)(R¹⁰)NC(Z), (R⁹)(R¹⁰)NC(Z)O, R⁸C(Z)N(R¹¹), R⁸OC(Z)N(R¹¹), (R⁹)(R¹⁰)NC(Z)N(R¹¹), (R⁹)(R¹⁰)NS(O)₂, R⁸S(O)₂N(R¹¹), (R⁹)(R¹⁰)NS(O)₂N(R¹¹), and R⁸SC(Z)N(R¹¹), wherein any perfluoro-C₁₋₆-alkyl, C₃₋₈-cycloalkyl, C₂₋₆-alkenyl, C₂₋₆-alkynyl, and C₃₋₈-heterocycloalkyl residue is optionally substituted in one or more positions independently of each other by a group selected from B, and also wherein any aryl and heteroaryl residue is optionally substituted in one or more positions independently of each other by a group selected from C;

B is defined as:

A, or a C₁₋₆-alkyl optionally substituted in one or more positions independently of each other by a group selected from D, provided that if B is directly attached to a double or to a triple bond, or to a carbon directly attached to a heteroatom, B is not HO, HS, R⁹HN, (R⁹)(R¹⁰)NC(Z)NH, (R⁹)(R¹⁰)NS(O)₂NH, or R⁶S(O)₂NH, and also provided that if B is directly attached to a double or to a triple bond, B is not Z;

C is defined as:

A, or a C₁₋₆-alkyl optionally substituted in one or more positions independently of each other by a group selected from D, provided that C is not Z;

D is selected from:

halogen, cyano, nitro, azido, Z, R⁸O, R⁸C(Z), R⁸C(Z)O, R⁸OC(Z), R⁸S, R⁸S(O), R⁸S(O)₂, R⁸S(O)₂O, R⁸OS(O)₂, (R⁹)(R¹⁰)N, (R⁹)(R¹⁰)NC(Z), (R⁹)(R¹⁰)NC(Z)N(R¹¹), (R⁹)(R¹⁰)NS(O)₂. R⁸S(O)₂N(R¹¹), and (R⁹)(R¹⁰)NS(O)₂N(R¹¹);

Y is selected from:

hydrogen, halogen, hydroxy, C₁₋₆-alkoxy, halo-C₁₋₆-alkyloxy, perfluoro-C₁₋₆-alkyloxy, C₁₋₆-acyloxy, C₁₋₆-alkylthio, halo-C₁₋₆alkylthio, perfluoro-C₁₋₆alkylthio, C₁₋₆-alkylsulphonyloxy, azido, and (R⁹)(R¹⁰)N;

Z is a substituent connected by a double bond, and is selected from:

O═, S═, R⁸N═, (R⁹)(R¹⁰)NN═, R⁸ON═, (R⁹)(R¹⁰)NS(O)₂N═, NCN═, O₂NCH═, and (R⁹)(R¹⁰)C═;

n is 0, 1, 2 or 3;

or pharmaceutically acceptable salts, stereoisomers or prodrugs thereof.

### DETAILED DESCRIPTION OF THE INVENTION

The present invention relates to compounds useful as glucocorticoid receptor modulators, and have the general formula I described above.

One embodiment of the present invention relates to compounds according to the general formula I, wherein R¹ is COOH or heteroaryl, preferably COOH.

Another embodiment of the present invention relates to compounds according to the general formula I, wherein R² and R³ are, independently of each other, halogen or C₁₋₆-alkyl, preferably both R² and R³ are halogen.

In a particularly preferred aspect both R² and R³ are bromine.

Another embodiment of the present invention relates to compounds according to the general formula I, wherein R⁴ is C₁₋₁₂-alkyl, C₃₋₈-cycloalkyl, C₂₋₆-alkenyl, C₂₋₆-alkynyl, C₃₋₈-heterocycloalkyl, halogen, (R⁹)(R¹⁰)N, or R⁸C(Z)N(R¹¹). Preferably, R⁴ is C₁₋₁₂-alkyl, halogen, (R⁹)(R¹⁰)N, or R⁸C(Z)N(R¹¹). More preferably, R⁴ is C₁₋₁₂-alkyl, and most preferably, R⁴ is isopropyl.

Another embodiment of the present invention relates to compounds according to the general formula I, wherein R⁵ is C₁₋₁₂-alkyl, C₃₋₈-cycloalkyl, C₃₋₈-heterocycloalkyl, aryl or heteroaryl, preferably wherein R⁵ is C₁₋₆-alkyl, C₃₋₈-cycloalkyl, aryl, or heteroaryl.

In a particularly preferred aspect R⁵ is aryl or heteroaryl.

Another embodiment of the present invention relates to compounds according to the general formula I, wherein R⁶ is C₁₋₁₂-alkyl, C₃₋₈-cycloalkyl, R⁸O, R⁸S, R⁸S(O), R⁸S(O)₂, (R⁹)(R¹⁰)N, R⁸C(Z)O, R⁸OC(Z)O, R⁸C(Z)N(R¹¹), R⁸OC(Z)N(R¹¹), R⁸S(O)ₙO, (R⁹)(R¹⁰)NC(Z)O, (R⁹)(R¹⁰)NS(O)₂O, R⁸S(O)₂N(R¹¹), and (R⁹)(R¹⁰)NS(O)₂N(R¹¹).

Preferably R⁶ is C₁₋₆-alkyl, R⁸O, R⁸S, (R⁹)(R¹⁰)N, R⁸C(Z)O, R⁸C(Z)N(R¹¹), or R⁸S(O)₂N(R¹¹).

In a particularly preferred aspect R⁶ is R⁸O, (R⁹)(R¹⁰)N, R⁸C(O)O, R⁸C(O)NH, or R⁸S(O)₂NH.

Another embodiment of the present invention relates to compounds according to the general formula I, wherein R⁷ is hydrogen, C₁₋₆alkyl, C₂₋₆-alkenyl, or C₂₋₆-alkynyl. Preferably R⁷ is hydrogen.

Another embodiment of the present invention relates to compounds according to the general formula I, wherein R⁶ is R⁸O, (R⁹)(R¹⁰)N, R⁸C(O)O, R⁸C(O)NH, or R⁸S(O)₂NH and R⁷ is hydrogen.

Another embodiment of the present invention relates to compounds according to the general formula I, wherein wherein R⁸, R⁹, R¹⁰, and R¹¹ are independently of each other hydrogen or C₁₋₆-alkyl, or R⁹ and R¹⁰ together with the nitrogen atom to which they are bound form a saturated heterocyclic ring having 5-6 ring members and which ring optionally contain 1 heteroatom, and which optionally is substituted by C₁₋₆-alkyl.

Another embodiment of the present invention relates to compounds according to the general formula I, wherein Y is hydroxy or C₁₋₆-alkoxy. Preferably Y is C₁₋₆-alkoxy.

Another embodiment of the present invention relates to compounds according to the general formula I, wherein n is 1 or 2. Preferably n is 1.

A preferred embodiment of the present invention relates to compounds according to the general formula I, wherein R¹ is COOH or heteroaryl; R² and R³ is independently of each other halogen or C₁₋₆-alkyl, or wherein both R² and R¹ are halogen; R⁴ is C₁₋₁₂-alkyl, halogen, (R⁹)(R¹⁰)N, or R⁸C(Z)N(R¹¹); R⁵ is C₁₋₆-alkyl, C₃₋₈-cycloalkyl, aryl, or heteroaryl; R⁶ is C₁₋₆-alkyl, R⁸O, R⁸S, (R⁹)(R¹⁰)N, R⁸C(Z)O, R⁸C(Z)N(R¹¹), or R⁸S(O)₂N(R¹¹); R⁷ is hydrogen; R⁸, R⁹, R¹⁰ and R¹¹ are independently of each other hydrogen or C₁₋₆-alkyl, or R⁹ and R¹⁰ together with the nitrogen atom to which they are bound form a ring having 5-6 ring members and which ring optionally contain 1 heteroatom, and which optionally is substituted by C₁₋₆-alkyl; Y is hydroxy or C₁₋₆alkoxy; and n is 1 or 2.

Another, more preferred embodiment of the present invention, relates to compounds according to the general formula I, wherein R¹ is COOH; R² and R³ is independently of each other halogen; R⁴ is C₁₋₁₂-alkyl; R⁵ is aryl or heteroaryl; R⁶ is R⁸O, R⁸S, (R⁹)(R¹⁰)N, R⁸C(O)O, R⁸C(O)NH, or R⁸S(O)₂NH; R⁷ is hydrogen; R⁸, R⁹, and R¹⁰ are independently of each other hydrogen or C₁₋₆-alkyl, or R⁹ and R¹⁰ together with the nitrogen atom to which they are bound form a ring having 5-6 ring members and which ring optionally contain 1 heteroatom, and which optionally is substituted by C₁₋₆-alkyl; Y is C₁₋₆alkoxy; and n is 1.

Compounds of the invention include, but are not limited to, the following:

3,5-Dibromo-4-[2-(1-hydroxyethyl)-5-isopropyl-4-methoxyphenoxy]phenylacetic acid (E1);

3,5-Dibromo-4-[2-(1-{3-indolyl}ethyl)-5-isopropyl-4-methoxyphenoxy]phenylacetic acid (E2);

4-[2-(2-Cyclopentyl-1-hydroxyethyl)-5-isopropyl-4-methoxyphenoxy]-3,5-dibromo-phenylacetic acid (E3);

3,5-Dibromo-4-{[2-(hydroxy(phenyl)methyl)]-5-isopropyl-4-methoxyphenoxy}phenylacetic acid (E4);

3,5-Dibromo-4-{5-isopropyl-4-methoxy-2-[(2-{methylsulfonyl)ethoxy(phenyl)methyl]-phenoxy}phenylacetic acid (E5);

3,5-Dibromo-4-{2-[hydroxy(2-methylphenyl)methyl]-5-isopropyl-4-methoxyphenoxy}-phenylacetic acid (E6);

3,5-Dibromo-4-{2-[(2,4-difluorophenoxy)(2-methylphenyl)methyl]-5-isopropyl-4-methoxyphenoxy}phenylacetic acid (E7);

3,5-Dibromo-4-{2-[2-butylamino(2-methylphenyl)methyl]-5-isopropyl-4-methoxyphenoxy}-phenylacetic acid (E8);

3,5-Dibromo-4-{2-[hydroxy(3-methylphenyl)methyl]-5-isopropyl-4-methoxy-phenoxy}-phenylacetic acid (E9);

3,5-Dibromo-4-[5-isopropyl-4-methoxy-2-(methoxy(3-methylphenyl)methyl)phenoxy]-phenylacetic acid (E10);

3,5-Dibromo-4-{2-[isopropoxy(3-methylphenyl)methyl]-5-isopropyl-4-methoxyphenoxy}-phenylacetic acid (E11);

4-{2-[Cyclohexyloxy(3-methylphenyl)methyl]-5-isopropyl-4-methoxyphenoxy}-3,5-dibromo-phenylacetic acid (E12);

3,5-Dibromo-4-{2-[(4-fluorophenoxy)(3-methylphenyl)methyl]-5-isopropyl-4-methoxyphenoxy}phenylacetic acid (E13);

3,5-Dibromo-4-{5-isopropyl-4-methoxy-2-[(4-methoxyphenoxy)(3-methylphenyl)-methyl]phenoxy}phenylacetic acid (E14);

3,5-Dibromo-4-{5-isopropyl-4-methoxy-2-[(3-methylphenyl)(4-nitrophenoxy)methyl]-phenoxy}phenylacetic acid (E15);

3,5-Dibromo-4-{2-[(4-aminophenoxy)(3-methylphenyl)methyl]-5-isopropyl-4-methoxyphenoxy}phenylacetic acid (E16);

3,5-Dibromo-4-{2-[(4-hydroxybenzoyloxy)(3-methylphenyl)methyl]-5-isopropyl-4-methoxy]-phenoxy}phenylacetic acid (E17);

4-{2-[Chloro(3-methylphenyl)methyl]-5-isopropyl-4-methoxyphenoxy}3,5-dibromo-phenylacetic acid (E18);

4-{2-[Amino(3-methylphenyl)methyl]-5-isopropyl-4-methoxyphenoxy}3,5-dibromo-phenylacetic acid (E19);

3,5-Dibromo-4-{2-[isopropylamino(3-methylphenyl)methyl]-5-isopropyl-4-methoxyphenoxy}phenylacetic acid (E20);

4-{2-[Cyclopropylamino(3-methylphenyl)methyl]-5-isopropyl-4-methoxyphenoxy}3,5-dibromo-phenylacetic acid (E21);

3,5-Dibromo-4-{2-[(1-pyrrolidino)(3-methylphenyl)methyl]-5-isopropyl-4-methoxyphenoxy}phenylacetic acid (E22);

3,5-Dibromo-4-{2-[(1-piperidino)(3-methylphenyl)methyl]-5-isopropyl-4-methoxyphenoxy}phenylacetic acid (E23);

3,5-Dibromo-4-{2-[(2-methoxy-1-ethyl)amino(3-methylphenyl)methyl]-5-isopropyl-4-methoxyphenoxy}phenylacetic acid (E24);

3,5-Dibromo-4-{2-[(2-{N,N-diethylamino}-1 ethyl)amino(3-methylphenyl)-methyl]-5-isopropyl-4-methoxyphenoxy}phenylacetic acid (E25);

3,5-Dibromo-4-{2-[(3-methylphenyl)(2-{1-piperidino}-1-ethyl)amino-methyl]-5-isopropyl-4-methoxyphenoxy}phenylacetic acid (E26);

3,5-Dibromo-4-{2-[(1-piperazino)(3-methylphenyl)methyl]-5-isopropyl-4-methoxyphenoxy}phenylacetic acid (E27);

3,5-Dibromo-4-{2-[4-methoxybenzylamino(3-methylphenyl)methyl]-5-isopropyl-4-methoxyphenoxy}phenylacetic acid (E28);

4-{2-[(3-Carboxyphenyl)amino(3-methylphenyl)methyl]-5-isopropyl-4-methoxyphenoxy}-3,5-dibromo-phenylacetic acid (E29);

3,5-Dibromo-4-{2-[(N-{4-hydroxybenzoyl}amino)(3-methylphenyl)methyl]-5-isopropyl-4-meth oxyphenoxy}phenylacetic acid (E30);

3,5-Dibromo-4-{2-[(N-{4-methylbenzenesulfonyl}amino)(3-methylphenyl)-methyl]-5-isopropyl-4-methoxyphenoxy}phenylacetic acid (E31);

4-{2-[Benzylthio(3-methylphenyl)methyl]-5-isopropyl-4-methoxyphenoxy}3,5-dibromo-phenylacetic acid (E32);

3,5-Dibromo-4-{2-[(2-furylmethylthio)(3-methylphenyl)methyl]-5-isopropyl-4-methoxyphenoxy}phenylacetic acid (E33);

4-{2-[Carboxymethylthio(3-methylphenyl)methyl]-5-isopropyl-4-methoxyphenoxy}3,5-dibromo-phenylacetic acid (E34);

3,5-Dibromo-4-{2-[(3-methylphenyl)phenylthio)methyl]-5-isopropyl-4-methoxyphenoxy}phenylacetic acid (E35);

3,5-Dibromo-4-{2-[hydroxy(4-methylphenyl)methyl]-5-isopropyl-4-methoxyphenoxy}phenylacetic acid (E36);

3,5-Dibromo-4-{2-[1-isopropoxy-1-(4-methylphenyl)methyl]-5-isopropyl-4-methoxyphenoxy}phenylacetic acid (E37);

3,5-Dibromo-4-{2-[(3-isopropylphenyl)hydroxymethyl]-5-isopropyl-4-methoxyphenoxy}-phenylacetic acid (E38);

3,5-Dibromo-4-{2-[(3-fluorophenyl)hydroxymethyl]-5-isopropyl-4-methoxyphenoxy}phenylacetic acid (E39);

3,5-Dibromo-4-{2-[hydroxy(3-iodopheryl)methyl]-5-isopropyl-4-methoxyphenoxy}phenylacetic acid (E40);

3,5-Dibromo-4-{2-[hydroxy(3-trifluorophenyl)methyl]-5-isopropyl-4-methoxyphenoxy}-phenylacetic acid (E41);

3,5-Dibromo-4-{5-isopropyl-4-methoxy-2-[methoxy-(3-trifluorophenyl)methyl]-phenoxy}phenylacetic acid (E42);

3,5-Dibromo-4-{2-[hydroxy(3-trifluoromethoxyphenyl)methyl]-5-isopropyl-4-methoxyphenoxy}phenylacetic acid (E43);

3,5-Dibromo-4-{2-[(3-dimethylaminophenyl)hydroxymethyl]-5-isopropyl-4-methoxyphenoxy}phenylacetic acid (E44);

3,5-Dibromo-4-{2-[(3,5-dimethylphenyl)hydroxymethyl]-5 isopropyl-4-methoxy-phenoxy}phen ylacetic acid (E45);

4-{2-[Cyclohexyloxy(3,5-dimethylphenyl)methyl]-5-isopropyl-4-methoxyphenoxy}3,5-dibromophenylacetic acid (E46);

3,5-Dibromo-4-{2-[(3,5-difluorophenyl)hydroxymethyl]-5-isopropyl-4-methoxyphenoxy}-phenylacetic acid (E47);

4-{[2-(3-chloro-2-fluorophenyl)hydroxymethyl]-5-isopropyl-4-methoxyphenoxy}3,5-dibromophenylacetic acid (E48);

4-{[2-(3-Chloro-2-fluorophenyl)methoxymethyl]-5-isopropyl-4-methoxyphenoxy}3,5-dibromo-phenylacetic acid (E49);

3,5-Dibromo-4-[2-(4-fluoro-3-methylphenyl)hydroxymethyl)-5-isopropyl-4-methoxyphenoxy]phenylacetic acid (E50);

4-{5-(2-Cyclopentytethyl)-2-[hydroxy(3-methylphenyl)methyl]4-methoxy-phenoxy}-3,5-dibro mophenylacetic acid (E51);

4-[2-[hydroxy(3-methylphenyl)methyl]-5-iodomethoxyphenoxy)3,5-dibromophenylacetic acid (E52);

4-{5-acetamido-2-[hydroxy(3-methylphenyl)methyl]4-methoxyphenoxy)3,5-dibromo-phenylacetic acid (E53);

4-{2-Hydroxy(3-methylphenyl)methyl]4-methoxy-5-(3-methylbenzamido)-phenoxy}3,5-dibromophenylacetic acid (E54);

3,5-Dibromo-4-{4-hydroxy-2-[hydroxy(3-methylphenyl)methyl]-5-isopropylphenoxy}phenylacetic acid (E55);

3,5-Dibromo-4-{2-[hydroxy(3-methylphenyl)methyl]isobutyloxy-5-isopropylphenoxy)-phenylacetic acid (E56);

3,5-Dibromo-4-{[2-fluoroethoxy]-2-[hydroxy-(3-methylphenyl)methyl]-5-isopropyl-phenoxy}phenylacetic acid (E57);

3,5-Dibromo-4-{2-[hydroxy(3-methylphenyl)methyl]-5-isopropyl-4-methoxyphenoxy}phenylpropionic acid (E58);

or pharmaceutically acceptable salts, stereoisomers or prodrugs thereof.

The present invention also relates to pharmaceutical compositions comprising any of the compounds of the present invention together with a pharmaceutically acceptable diluent or carrier.

The present invention also relates to processes for making the pharmaceutical compositions of the present invention.

Another embodiment of the invention is a method preventing, inhibiting or treating a disease associated with a metabolic dysfunction by administering to the mammal a therapeutically effective amount of any of the compounds or pharmaceutical compositions described herein.

Another embodiment of the invention is a method preventing, inhibiting or treating a disease, which is dependent on the expression of a glucocorticoid receptor regulated gene, by administering to the mammal a therapeutically effective amount of any of the compounds or pharmaceutical compositions described herein.

The diseases referred to comprise, but are not limited to Type 1 insulin dependent diabetes, Type 2 non-insulin dependent diabetes, Cushing's syndrome, inflammation, autoimmune disease, transplant rejection, neoplasm, leukemia, lymphoma, Cushings disease, adrenal disease, renal disease, cerebrovascular ischemia, hypercalcemia, cerebral edema, thrombocytopenia, inflammatory bowel disease, wound healing, HIV infection, central nervous system disease, spinal cord tumour, glaucoma, sleep disorder, depression, anxiety disorder, atherosclerosis, hypertension, osteoporosis, occular hypertension, nephrotoxicity, infarction, endometriosis, pregnancy disorder, psychosis, Alzheimers disease, cocaine use disorder, asthma, allergic rhinitis, conjuctivitis, rheumatoid arthritis, dermatitis, eczema, osteoarthritis, hypoglycemia, hyperinsulinemia, hyperlipidemia and obesity.

Another embodiment of the invention is a method of eliciting a glucocorticoid receptor modulating effect in a mammal in need thereof, comprising administering to the mammal a therapeutically effective amount of any of the compounds or any of the pharmaceutical compositions described herein.

One aspect of this embodiment is the method wherein the glucocorticoid receptor modulating effect is an antagonizing effect.

The compounds of the invention are glucocorticoid receptor antagonists that are preferably liver selective, and as such may be useful in the treatment of diabetes (alone or in combination with agents that stimulate insulin release such as sulfonylureas, influence liver glucose production such as metformin, affect the sensitivity to insulin such as troglitazone, or inhibit glucose absorption such as α-glucosidase inhibitors).

The compounds of the present invention in labelled form, e.g. isotopically labelled, may be used as diagnostic agents.

Further exemplifying the invention is the use of any of the compounds described above in the manufacture or preparation of a medicament for therapeutic treatment or prevention of a disease associated with a metabolism dysfunction, or a disease which is dependent on the expression of a glucocorticoid receptor regulated gene, in a mammal in need thereof. Still further exemplifying the invention is the use of any compounds described above in the manufacture or preparation of a medicament for the therapeutic treatment or prevention of Type 1 insulin dependent diabetes, Type 2 non-insulin dependent diabetes, Cushing's syndrome, inflammation, autoimmune disease, transplant rejection, neoplasm, leukemia, lymphoma, Cushings disease, adrenal disease, renal disease, cerebrovascular ischemia, hypercalcemia, cerebral edema, thrombocytopenia, inflammatory bowel disease, wound healing, HIV infection, central nervous system disease, spinal cord tumour, glaucoma, sleep disorder, depression, anxiety disorder, atherosclerosis, hypertension, osteoporosis, occular hypertension, nephrotoxicity, infarction, endometriosis, pregnancy disorder, psychosis, Alzheimers disease, cocaine use disorder, asthma, allergic rhinitis, conjuctivitis, rheumatoid arthritis, dermatitis, eczema, osteoarthritis, hypoglycemia, hyperinsulinemia, hyperlipidemia and obesity.

The compounds of the present invention can be administered in such oral dosage forms as tablets, capsules (each of which includes sustained release or timed release formulations), pills, powder, granules, elixirs, tinctures, suspensions, syrups and emulsions. Likewise, they may also be administered in intravenous (bolus or infusion), intraperitoneal, topical (e.g. ocular eyedrop), subcutaneous, intramuscular, or transdermal (e.g. patch) form, all using forms well known to those of ordinary skill in the pharmaceutical arts.

The dosage regimen utilizing the compounds of the present invention is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches or iontophoretic devices well known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, exipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol, and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms includes sodium oleate, sodium stearate, magnesium stearate, sodium benzoate, sodium acetate, sodium chloride, and the like. Disintegrators include without limitation starch, methylcellulose, agar, bentonite, xanthan gum, and the like.

The compounds of the present invention can also be administered in the form of liposome delivery systems, such as small unilamellar vesicles, large unilamellar vesicles and multilamellar vesicles. Liposomes can be formed from e.g. phospholipids, cholesterol, stearylamine, or phosphatidylcholines.

The following definitions apply to the terms as used throughout this specification, unless otherwise limited in specific instances.

The term “GR antagonist” as used herein is intended to cover any moiety that binds to a glucocorticoid receptor, or a complex of which a glucocorticoid receptor forms a part, and acts as an antagonist or a partial antagonist.

The term “halogen” and “halo”, as used herein alone or as part of another group, refers to chlorine, bromine, fluorine, and iodine.

The term “heteroatom” and “hetero”, as used herein, refers to nitrogen, oxygen, sulphur, and in heterocyclic rings, also selenium.

The term C₁₋₆-alkyl, as used herein alone or as part of another group, refers to an alkyl group which may be straight or branched. Exemplary C₁₋₆-alkyl groups include, but are not restricted to, methyl, ethyl, propyl, isopropyl, butyl, sec-butyl, tert-butyl, pentyl, isopentyl, hexyl, and isohexyl.

The term C₁₋₁₂-alkyl, as used herein alone or as part of another group, refers to an alkyl group which may be straight or branched. Exemplary C₁₋₁₂-alkyl groups include, but are not restricted to, methyl, ethyl, propyl, isopropyl, butyl, sec-butyl, tert-butyl, pentyl, isopentyl, neopentyl, hexyl, isohexyl, heptyl, octyl, isooctyl, nonyl, decyl, isodecyl, undecyl, and dodecyl.

The term C₃₋₈-cycloalkyl, as used herein alone or as part of another group, refers to a mono-, or bicyclic alkyl group, which may contain one or more unsaturations (double, and/or triple bonds). Exemplary C₃₋₈-cycloalkyl groups include, but are not restricted to, cyclopropyl, cyclobutyl, cyclopentyl, cyclohexyl, cycloheptyl, cyclooctyl, cyclopentenyl, cyclohexenyl, cycloheptenyl, cyclooctenyl, cyclooctynyl, bicycloheptyl, bicyclooctyl, and bicyclooctenyl. It is also understood that a single carbon of the C₃₋₈-acycloalkyl may be common to another C₃₋₈-cycloalkyl or C₃₋₈-heterocycloalkyl, forming a so called spiro-compound.

The term C₁₋₆-alkoxy, as used herein alone or as part of another group, refers to an alkoxy group which may be straight or branched. Exemplary C₁₋₆-alkoxy groups include, but are not restricted to, methoxy, ethoxy, propoxy, isopropoxy, butoxy, sec-butoxy, tert-butoxy, pentyloxy, isopentyloxy, hexyloxy, and isohexyloxy.

The term C₂₋₆-alkenyl, as used herein alone or as part of another group, refers to an alkenyl group which may be straight or branched. Exemplary C₂₋₆-alkenyl groups include, but are not restricted to, vinyl, 1-propenyl, 2-propenyl, propadienyl, 1-butenyl, 2-butenyl, 3-butenyl, 1,3-butadienyl, 1-pentenyl, 2-pentenyl, 4-pentenyl, and 5-hexenyl.

The term C₂₋₆-alkynyl, as used herein alone or as part of another group, refers to an alkynyl group which may be straight or branched. Exemplary C₂₋₆-alkynyl groups include, but are not restricted to, ethynyl, 1-propynyl, 2-propynyl, 1-butynyl, 2-butynyl, 3-butynyl, 1-pentynyl, 2-butynyl, 4-pentynyl, 1-hexynyl, 3-hexynyl, and 5-hexynyl.

The term C₃₋₈-heterocycloalkyl, as used herein alone or as part of another group, refers to a mono-, or bicyclic alkyl group which may contain one or more heteroatoms, and which may contain one or more unsaturations (double, and/or triple bonds). Exemplary C₃₋₈-heterocycloalkyl groups include, but are not restricted to, aziridine, azetidine, pyrrolidine, pyrroline, piperidine, tetrahydropyridine, dihydropyridine, pyrazolidine, imidazolidine, imidazoline, piperazine, morpholine, thiomorpholine, oxirane, oxetane, tetrahydrofuran, tetrahydropyran, dihydropyran, 1,3-dioxolan, 1,3-dioxane, 1,4-dioxane, thiirane, thietane, thiolane, 1,3-dithiolane, 1,4-dithiane, 1,3,5-trithiane, quinuclidine, and tropane. It is also understood that a single carbon or nitrogen of the C₃₋₈--heterocycloalkyl may be common to another C₃₋₈-cycloalkyl-, or C₃₋₈-heterocycloalkyl-group, forming a so called spiro-compound.

Halo-C₁₋₆-alkyl groups may be straight or branched and have one, two or three halogens. Exemplary halo-C₁₋₆-alkyl groups include, but are not restricted to, fluoromethyl, difluoromethyl, chlorofluoromethyl, dichlorofluoromethyl, chlorodifluoromethyl, dichloromethyl, trichloromethyl, 1-fluoroethyl, 2-fluoroethyl, 1,1-difluoroethyl, 1,2-difluoroethyl, 2,3-difluoroethyl, 2,2,2-trifluoroethyl, 2-chloroethyl, 1,1,1-trifluoropropyl, 1-chloropropyl, 3-chloropropyl, 2,3-dichloropropyl, 6,6,6-trifluorohexyl, and 6-chlorohexyl.

Hydroxy-C₁₋₆-alkyl groups may be straight or branched and have one or two hydroxy groups, provided that if two hydroxy groups are present, they are not both attached to the same carbon atom. Exemplary hydroxyalkyl groups include, but are not restricted to, hydroxymethyl, 1-hydroxyethyl, 2-hydroxyethyl, 2-hydroxypropyl, 3-hydroxypropyl, 2-hydroxybutyl, 3-hydroxybutyl, 4-hydroxybutyl, and 2,3-dihydroxypropyl.

The term perfluoro-C₁₋₆-alkyl, as used herein alone or as part of another group, refers to an C₁₋₆-lalkyl group in which all hydrogens are replaced by fluorines. Exemplary perfluoro-C₁₋₆-alkyl groups include, but are not restricted to, trifluoromethyl, pentafluoroethyl, heptafluoropropyl and heptafluoroisopropyl.

The term perfluoro-C₁₋₆-alkoxy, as used herein alone or as part of another group, refers to an Clmalkoxy group in which all hydrogens are replaced by fluorines. Exemplary perfluoro-C₁₋₆-alkoxy groups include, but are not restricted to, trifluoromethoxy, pentafluoroethoxy, heptafluoropropoxy, and heptafluoroisopropoxy.

The term aryl is intended to include monocyclic or bicyclic ring systems having from 6 to 10 ring carbon atoms, in which at least one ring is aromatic. Examples of such ring systems are benzene, naphtalene, 1,2,3,4-tetrahydronaphtalene, indan, and indene.

The term heteroaryl refers to a mono-, bi- or tricyclic ring system having from 5 to 10 ring atoms, in which at least one ring is aromatic, and in which one or more of the ring atoms are other than carbon, such as nitrogen, sulphur, oxygen and selenium. Examples of such heteroaryl rings include, but are not restricted to, pyrrole, furan, thiophene, pyrazole, imidazole, oxazole, isoxazole, thiazole, isothiazole, 1,2,3-triazole, 1,2,4-triazole, 1,3,4-triazole, 1,2,3-oxadiazole, 1,2,4-oxadiazole, 1,3,4-oxadiazole, 1,2,3-thadiazole, 1,2,4-thiadiazole, 1,3,4-thiadiazole, tetrazole, pyridine, indole, isoindole, indoline, isoindoline, quinoline, 1,2,3,4-tetrahydroquinoline, isoquinoline, 1,2,3,4-tetrahydroisoquinoline, quinolizine, carbazole, acridine, benzofuran, isobenzofuran, chroman, isochroman, benzothiophene, pyridazine, pyrimidine, pyrazine, indazole, benzimidazole, cinnoline, quinazoline, quinoxaline, phthalazine, 1,5-naphthyridine, 1,8-naphthyridine, phenazine, benzoxazole, 3,4-dihydro-2H-1,4-benzoxazine, benzothiazole, phenothiazine, 1,3-benzodioxole, benzodioxane, 2,1,3-benzoxadiazole, 2,1,3-benzothiazole, 2,1,3-benzoselenadiazole, purine, and pteridine. The ring system may be linked to the rest of the molecule via a carbon or nitrogen atom thereof.

The compounds of formula I in the invention may contain at least one chiral center and may therefore exist as optical isomers. The invention therefore comprises optically inactive racemic (rac) mixtures (a one to one mixture of enantiomers), optically enriched scalemic mixtures as well as optically pure individual enantiomers. The compounds in the invention also may contain more than one chiral center and therefore may exist as diastereomers; The invention therefore comprises individual diastereomers as well as any mixture of diastereomers.

The compound of formula I in the invention may contain geometrical isomers and may therefore exist as either the E (entgegen) or Z (zusammen) isomers. The invention therefore comprises individual E or Z isomers as well as any mixture of E and Z isomers.

The compound of formula I in the invention may exist in tautomeric forms, the invention therefore comprises the individual tautomeric forms as well as any mixture thereof.

The compounds of formula I can be present as salts, in particular pharmaceutically acceptable salts. If the compounds of formula I have, for example, at least one basic center, they can form acid addition salts. These are formed, for example, with inorganic acids, such as mineral acids, for example sulfuric acid, phosphoric acid or a hydrohalic acid, with organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as C₁₋₆-valkyl- or aryl-sulfonic acids which are unsubstituted or substituted, for example by halogen, for example methane- or ptoluenesulfonic acid. Corresponding acid addition salts can also be formed having, if desired, an additionally present basic center. The compounds of formula I having at least one acid group (for example COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or tri-ethanolamine. Corresponding internal salts may furthermore be formed. Salts which are unsuitable for pharmaceutical uses but which can be employed, for example, for the isolation or purification of free compounds I or their pharmaceutically acceptable salts, are also included.

Preferred salts of the compounds of formula I which include a basic group include monohydrochloride, hydrogensulfate, tartrate, fumarate or maleate.

Preferred salts of the compounds of formula I which include an acid group include sodium, potassium and magnesium salts and pharmaceutically acceptable organic amines.

Also included within the scope of the invention are polymorphs, hydrates, and solvates of the compounds of the instant invention.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example in “Design of Prodrugs” ed. H. Bundgaard, Elsevier, 1985, which is incorporated by reference herein in its entirety.

The present invention includes within its scope metabolites of compounds of formula I. Metabolites of the compounds includes active species produced upon introduction of compounds of this invention into the biological milieu.

The present invention includes within its scope compounds of formula I in isotopically labelled form.

The compounds of formula I may be prepared by the exemplary processes described in the following reaction schemes. Exemplary reagents and procedures for these reactions appear hereinafter and in the working Examples.

Compounds of formula I of the invention may be prepared using the sequence of steps outlined in Schemes 1 to 6 set out below. The groups R¹, R², R³, R⁴, R⁵, R⁶, R⁷, R⁸, R⁹, R¹⁰, and R¹¹ in Schemes 1 to 6 are as defined in formula I. The groups R¹, R², R³, R⁴, R⁵, R⁶, R⁷, R⁸, R⁹, R¹⁰, and R¹¹ may be modified one or several times, after or during the preparation of compounds of formula I by methods known in the art. Examples of such methods include, but are not restricted to, substitutions, reductions, oxidations, alkylations, hydrolysis, esterifications, and etherifications. Y′ is Y, or a group that can be transformed to Y in one or several synthetic steps, after or during the preparation of compounds of formula I, by methods known by a person skilled in the art. Examples of such transformations include, but are not restricted to, demethylation of methyl ethers, alkylations, silylations or acylations of a hydroxy group, hydrolysis of esters, carbamates, or silyl ethers, and transition metal catalyzed alkoxylations or aminations of triflates, chlorides, bromides, iodides, or boronic acids or esters. E is R¹, or a group that can be transformed to R¹ in one or several synthetic steps after or during the preparation of compounds of formula I, by methods known by a person skilled in the art. Examples of such transformations include, but are not restricted to, nucleophilic substitutions of an alkyl group activated by a halogen or a sulphonic acid ester, hydrolysis of an ester or a nitrile to give a carboxylic acid, or the transformation of a nitrile into an amide or into a tetrazole.

Scheme 1 describes a synthetic route that begins with a coupling reaction between an appropriately substituted iodonium salt 2 and an appropriately substituted phenol 1 to give the diarylether 3. The coupling is preferably catalyzed by metals, preferably Cu, Ni, Pd, or suitable salts, complexes, oxides or hydroxides thereof in the presence of a base. Suitable bases include, but are not restricted to, triethylamine, pyridine, K₂CO₃ and Cs₂CO₃. Alternatives to this ether formation include, but is not restricted to, transition metal catalyzed couplings of phenols with aryl halides or arylboronic acids, or other reactions known by a person skilled in the art. In the next step diarylether 3 is converted to the ketone 4 by introducing R⁵CO, e.g. by a Friedel-Crafts reaction of diarylether 3 with an appropriate acyl halid, carboxylic acid anhydride, carboxylic acid, or ketene. The reactiQn is preferably performed in the presence of a Lewis or a Brønstedt acid. Suitable acids include, but are not restricted to, H₂SO₄, polyphosphoric acid, CF₃SO₃H, TiC4, AlCl₃, ZnCl₂, BF₃OEt₂, and the like. Compound 5 is formed by introducing the group R⁶ into ketone 4 by a reaction with a nucleophile, e.g. a Grignard, organocerium, or an organolithium reagent, or in case R⁶ is hydrogen, by a reducing agent such as NaBH₄, NaBH₃CN, or diisobutylaluminium hydride. The hydroxy group of alcohol 5 may then be converted in one ore several synthetic steps to the R⁷-group in compound 6. Examples of such conversions include, but are not restricted to, alkylations, acylations, halogenations, and reactions with alcohols or phenols in the presence of a Lewis or a Brønstedt acid. Suitable acids include, but are not restricted to H₂SO₄, polyphosphoric acid, CF₃SO₃H, TiCl₄, AlCl₃, BF₃.OEt₂, SnCl₂.H₂O, p-toluenesulfonic acid, and the like. If E is not R¹ in compound 6, E in compound 6 is transformed to R¹ in compound 7. Such transformations may be performed in one or several synthetic steps and include, but are not restricted to, nucleophilic substitutions (e.g. by cyanides) of an alkyl group activated by a halogen or a sulphonic acid ester (e.g. by mesylate, tosylate or triflate), hydrolysis of an ester or a nitrile to give a carboxylic acid, or the transformation of a nitrile into a tetrazole. Alternatively, alcohol 5 can be transformed to compound 8, using the same method as described above for the transformation of compound 6 to compound 7. Compound 8 may then be converted to 7 using the same method as described above for the transformation of alcohol 5 to compound 6. If Y′═Y, compound 7 is equal to compounds of formula I of the present invention, if not, Y′ is converted to Y as described previously.

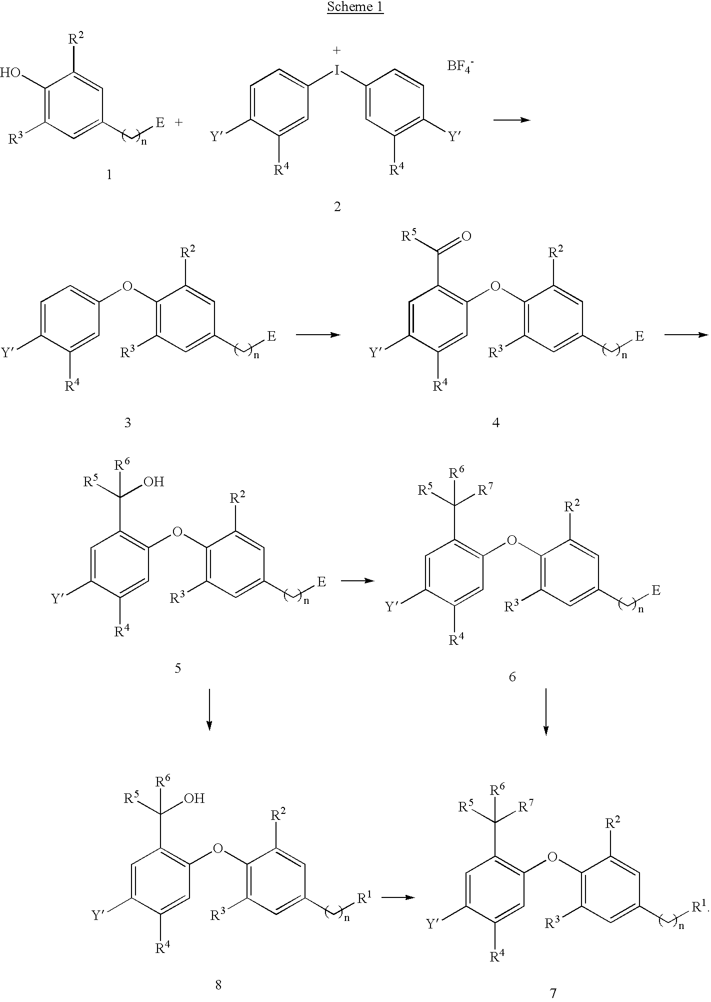

Scheme 2 describes an alternative synthesis to the intermediate 3 in Scheme 1. As in Scheme 1, the synthetic route begins with a coupling reaction, in this case between the iodonium salt 9 and the phenol 1 to give the diarylether 10. The coupling is preferably catalyzed by metals, preferably Cu, Ni, Pd, or suitable salts, complexes, oxides or hydroxides thereof in the presence of a base. Suitable bases include, but are not restricted to, triethylamine, pyridine, K₂CO₃ and Cs₂CO₃. Alternatives to this ether formation include, but is not restricted to, transition metal catalyzed couplings of phenols with aryl halides or arylboronic acids, or other reactions known by a person skilled in the art. In the next step diarylether 10 is converted to the ketone 11 by introducing RACO, e.g. by a Friedel-Crafts reaction of diarylether 10 with an appropriate acyl halide, carboxylic acid anhydride, carboxylic acid, or ketene. The reaction is preferably performed in the presence of a Lewis or a Brønstedt acid. Suitable acids include, but are not restricted to, H₂SO₄, polyphosphoric acid, CF₃SO₃H, TiCl₄, AlCl₃, ZnCl₂, BF₃.OEt₂, and the like. Compound 11 is converted to compound 12 in one or several synthetic transformations by reactions including, but not restricted to, nucleophilic substitutions, reductions, olefinations, oxidations, alkylations, hydrolysis, esterifications and etherifications. When R^{A}, R^{B} and R^{C}, together with the carbon to which they are bound equal R⁴, compound 12 is the same as compound 3, that may be transformed to compounds of formula I of the invention by using the synthetic sequences described in Scheme 1. If R^{A}, R^{B} and R^{C}, together with the carbon to which they are bound do not equal R⁴, they can be converted to R⁴ by reactions known by a person skilled in the art.

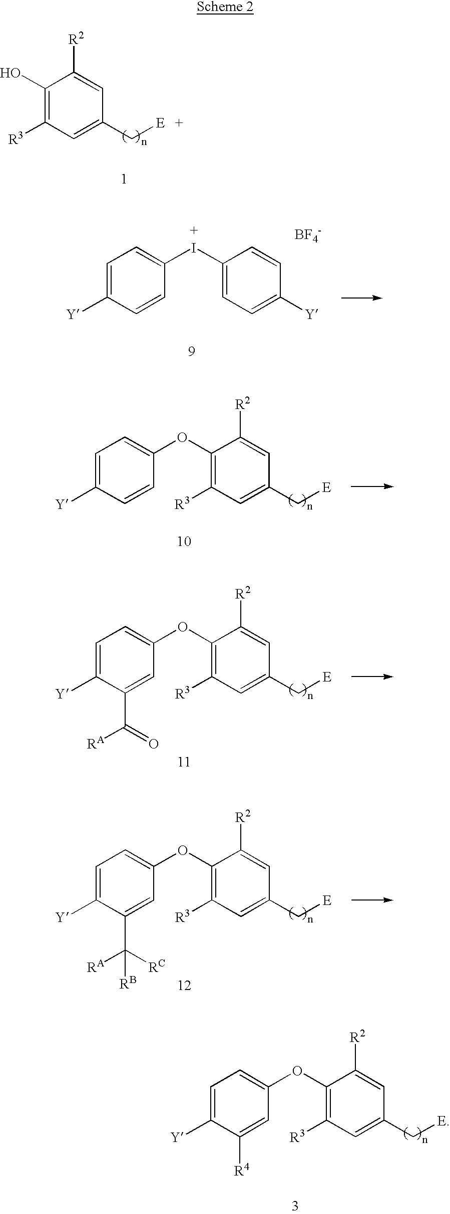

The R^{A}CO-group can also be introduced by a so called Fries rearrangement, as described in Scheme 3. In this case ester 13 (=10, where Y′═R^{A}COO) is converted to phenol 14 (=11, where Y′═OH) in a reaction catalyzed by light or by a Lewis or a Brønstedt acid. Suitable acids include, but are not restricted to, H₂SO₄, polyphosphoric acid, CF₃SO₃H, TiC4, AlCl₃, ZnCl₂, BF₃.OEt₂, and the like. Compound 13 can be prepared from compounds 9 or 10 by synthetic transformations known to those skilled in the art.

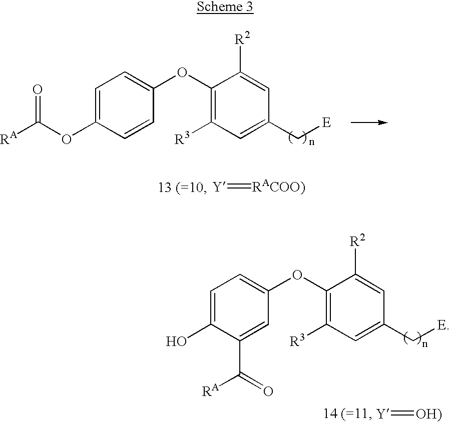

Scheme 4 describes yet another approach to introduce R⁴ into the molecule. The diarylether 10 is halogenated to give compound 15 (X=halogen). Suitable halogenating agents include, but are not restricted to iodine, NaI/NaOH, bromine, N-bromosuccinimide and 1,3-dibromo-5,5-dimethylhydantoin. Compound 15 is converted to compound 3 using one or several synthetic transformations. Such synthetic transformations include, but are not restricted to, transition metal catalyzed alkylations, alkenylations, alkynylations, arylatons, carbonylations, alkoxylations, and aminations. Scheme 4 also describes a sequence where the diarylether 10 is nitrated to give compound 15 (X═NO₂). The nitro group is then transformed in one or several synthetic steps, including, but not restricted to, reductions, alkylations, acylations, diazotations, halogenations, cyanation, and transition metal catalyzed reactions (as described above) to R⁴ in compound 3.

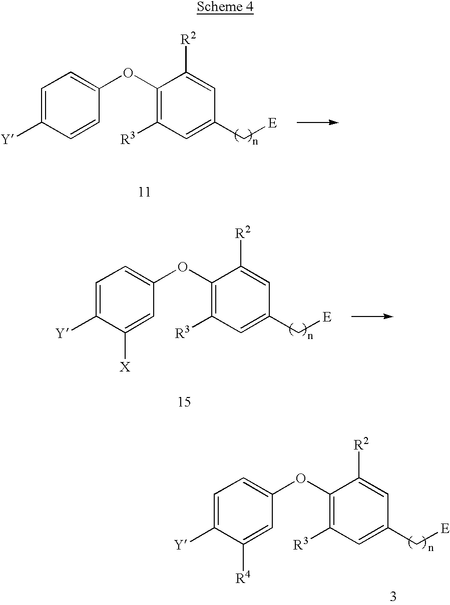

Scheme 5 depicts an alternative route to introduce RW into the molecule when R⁷ in compounds of formula 1 equals hydrogen. The aldehyde 16 (i.e. 4 where R⁵═H) is synthesized by formylation of compound 3 by reactions including, but not restricted to, electrophilic substitution reactions. Suitable electrophilic substitution reactions include, but are not restricted to the Vilsmeier reaction (i.e. a combination of an activating agent such as POCl₃, COCl₂ or (COCl)₂ and an amide such as DMF or N-phenyl-N-methylformamide) or the combination of MeOCHCl₂ and TiCl₄. The R⁵-group (R⁵ in alcohol 17 (i.e. 5 where R⁶═H) is introduced by a reaction of the aldehyde 16 and a nucleophile. Suitable nucleophiles include, but are not restricted to Grignard, organocerium, or organolithium reagents. If E is not R¹ in compound 17, it is transformed to R¹ in compound 18 by methods described previously. If Y′═Y, compound 18 (i.e. 8 where R⁶═H) is equal to compounds of formula I of the present invention where R⁶═OH and R⁷═H, if not, Y′ is converted to Y as described previously.

Analogously to the reactions described in Scheme 1, compound 19 (i.e. 7 where R⁷═H) may be obtained by transforming the hydroxy group in 18 to R⁶-groups other then hydroxy by synthetic transformations known to those skilled in the art. These synthetic transformations include, but are not restricted to, reactions with acylating or sulfonylating reagents (giving compounds of formula I of the present invention where R⁶═R⁵C(Z)O, R⁸OC(Z)O, R⁸S(O)ₙO, (R⁹)(R¹⁰)NC(Z)O, or (R⁹)(R¹⁰)NS(O)₂O), and R⁷═H), and reactions with alcohols, phenols, carboxylic acids, or amides in the presence of a suitable Lewis or Brønstedt acid, (giving compounds of formula I of the present invention where R⁶═R⁸C(O)O, or R⁸C(O)N(R¹¹) and R⁷═H).

The hydroxy group in 18 may also be converted to a halogen, using reagents such as HCl, HBr, SOCl₂, SOBr₂, or CBr₄/Ph₃P, giving compounds of formula I of the present invention where R⁶=halogen and R⁷═H. These halogenated compounds may in turn be transformed to compounds of formula I of the present invention where R⁶═R⁸O, R⁸S, (R⁹(R¹⁰)N, R⁸C(Z)O, R⁸C(Z)N(R¹¹), R⁸OC(Z)N(R¹¹), R⁸S(O)₂N(R¹¹) or (R⁹)(R¹⁰)NS(O)₂N(R¹¹), and R⁷═H, by a reaction with an appropriate nucleophilic reagent such as an alcohol, phenol, thiol, amine, carboxylic acid, amide, carbamate, sulfonamide, or a sulfamide. The reaction may be performed in the presence of a suitable base. Suitable bases include, but are not restricted to, triethylamine, N-ethyldiisopropylamine, K₂CO₃ and Cs₂CO₃.

As described in Scheme 5, an alternative to the synthetic sequences 17″ is the sequence 17″, where the transformation of 17′ equals the transformation of 18′, and the transformation of 20′ equals the transformation of 17′. When Y′═Y, compound 19 is equal to compounds of formula I of the present invention where R⁷═H, if not, Y′ is converted to Y as described previously.

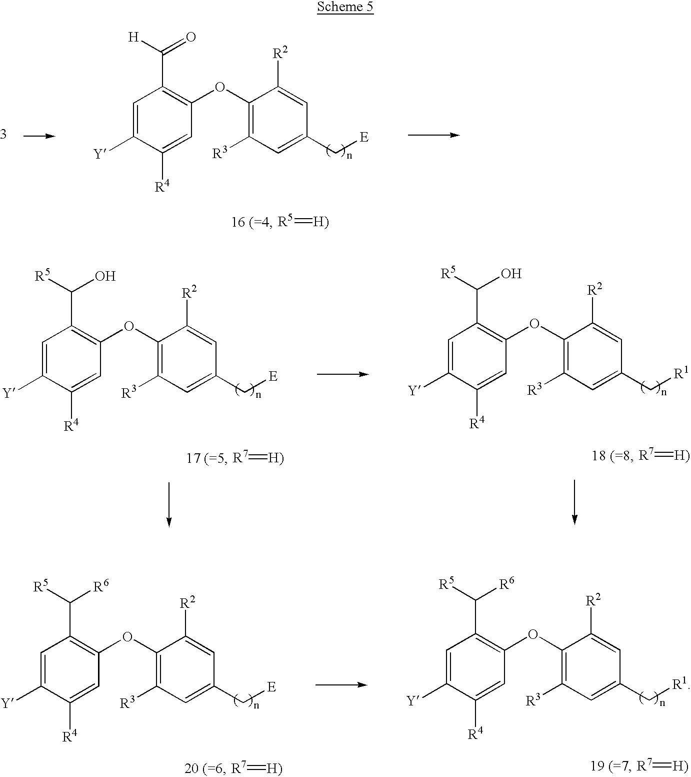

Compounds of the formula I, where R⁷═H, can also be obtained by the route described in Scheme 6. In this route, the R⁶ substituent is introduced prior to R⁵. Compound 21 is prepared analogously to compound 4 in Scheme I from compound 3. The substituent R⁶ is in this case selected from C₁₋₁₂-alkyl, perfluoro-C₁₋₆-alkyl, C₃₋₈-cycloalkyl, C₂₋₆-alkenyl, and C₂₋₆-alkynyl, and R¹ is aryl or heteroaryl. Reduction of the carbonyl group in compound 21, by methods described previously, affords the alcohol 22; which is allowed to react with electrophilic aromatic (e.g. phenols, arylethers, or anilines), or hetero-aromatic compounds (e.g. indoles or pyrroles), in the presence of a suitable Lewis or Brønstedt acid to give 23. When E is R¹, and Y′═Y, compound 23 is equal to compounds of formula I of the present invention where R⁷═H, if not, E is converted to R¹, and Y′ is converted to Y as described previously.

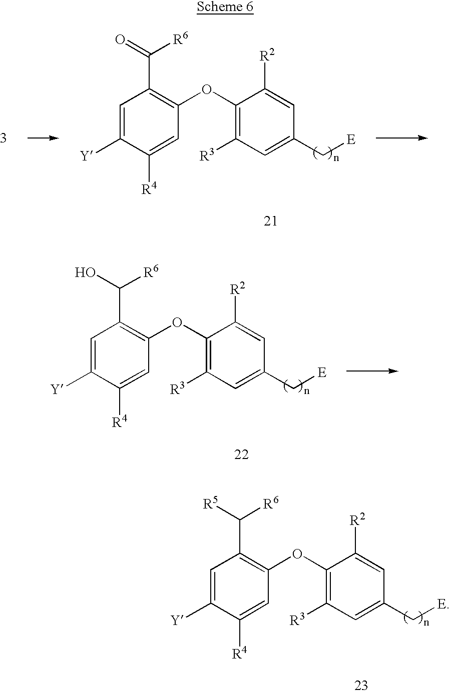

Those skilled in the art will readily understand that known variations of the processes described herein and of the experimental conditions, such as solvents, temperatures and times, of the following preparative procedures, can be used to prepare compounds of the formula I of the present invention. The following Examples represent preferred but non-limiting embodiments of the present invention.

### EXAMPLE 1

### 3,5-Dibromo-4-[2-(1-hydroxyethyl)-5-isopropyl-4-methoxgphenoxy]phenylacetic Acid (E1)

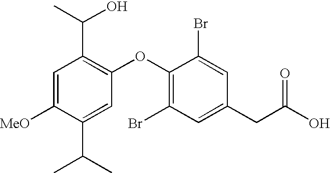

Step 1.

Bis(3-isopropyl-4-methoxyphenyl)iodonium Tetrafluoroborate.

Fuming nitric acid (22.3 ml, 477 mmol) was added dropwise to 30.8 mL of acetic anhydride cooled in a dry ice/CCl₄ bath. Iodine (10.3 g, 40.6 mmol) was added in one portion followed by dropwise addition of trifluoroacetic acid (37.9 mL, 492 mmol). The mixture was stirred at room temperature until the iodine was dissolved and then purged with N₂ to remove nitrogen oxides. The mixture was concentrated, the residue dissolved in acetic anhydride (115 mL) and cooled in a dry ice/CCl₄ bath. A solution of 2-isopropylanisole (30 g, 200 mmol) in acetic anhydride (138 mL) and trifluoroacetic acid (20.5 mL) was added dropwise with stirring. The mixture was left at room temperature overnight and concentrated. The residue was taken up into MeOH (138 mL) and treated with 10% aqueous NaHSO₃ (138 mL) and 2M aqueous NaBF₄ (0.92 L). After the precipitate had aggregated, petroleum ether was added and the supernatant was decanted. The precipitate was triturated with petroleum ether, filtered, washed with petroleum ether and dried at room temperature under vacuum to afford 14.7 g (71%) of the title compound.

Step 2.

3,5-Dibromo-4-(3-isopropyl-4-methoxyphenoxy)phenylacetic Acid Methyl Ester.

A solution of 3,5-dibromo-4-hydroxyphenylacetic acid methyl ester (5.27 g, 17.0 mmol) and triethylamine (1.89 g, 18.7 mmol) in CH₂Cl₂ (25 mL) was added dropwise to a mixture of bis(3-isopropyl-4-methoxyphenyl)iodonium tetrafluoroborate (13.0 g, 25.5 mmol) and copper bronze (2.14 g, 33.7 mmol) in CH₂Cl₂ (38 mL) at 0° C. The mixture was stirred in the dark for 4d and filtered through celite. The filtrate was concentrated and the residue purified by chromatography on silica gel (petroleum ether/EtOAc, 98:2) to give 6.0 g (76%) of the title compound.

Step 3.

4(2-Acetyl-5-isopropyl-4-methoxyphenoxy)-3,5-dibromophenylacetic Acid Methyl Ester.

TiCl₄ (12.8 g, 67.4 mmol) was added dropwise to a solution of 3,5-dibromo-4-(3-isopropyl-4-methoxyphenoxy)phenylacetic acid methyl ester (6.4 g, 13.5 mmol) and acetylchloride (2.6 g, 33.7 mmol) in CH₂Cl₂ (100 mL) at 0° C. The mixture was stirred at room temperature for 72 h, poured into ice and stirred for another hour. The layers were separated and the aqueous phase extracted twice with EtOAc. The combined extracts were dried over MgSO₄, concentrated and purified by chromatography to give 5,3 g (76%) of the title compound.

Step 4.

3,5-Dibromo-4-[2-(1-hydroxyethyl)-5-isopropyl-4-methoxyphenoxy]phenylacetic Acid.

NaBH₄ (90 mg, 2.38 mmol) was added in one portion to stirred mixture of 4-(2-acetyl-5-isopropyl-4-methoxyphenoxy)-3,5-dibromophenylacetic acid methyl ester (120 mg, 0.23 mmol), MeOH (4 mL) and 1M LiOH (1 mL) at room temperature for 16 h. The mixture was stirred for 3 h and acidified with 2M HCl. The mixture was concentrated and the remaining aqueous phase was extracted twice with EtOAc. The combined extracts were dried over MgSO₄ and concentrated. The residue was dissolved in MeOH and filtered through silica gel to give 107 mg (91%) of the title compound as a white solid. ¹H NMR (CDCl₃): δ 7.55 (s, 2H), 7.02 (s, 1H), 6.10 (s, 1H), 5.39 (q, 1H), 3.82 (s, 3H), 3.65 (s, 2H), 3.18 (m, 1H), 1.63 (d, 2H), 1.02 (dd, 6H).

### EXAMPLE 2

### 3,5-Dibromo-4-[2-(1-{3-indolyl}ethyl)-5-isopropyl-4-methoxyphenoxy]phenylacetic Acid (E2)

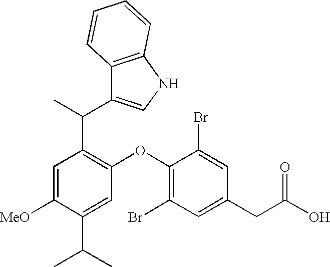

Indole (11 mg, 0.1 mmol) followed by tin(II)chloride dihydrate (21 mg, 0.1 mmol) was added to a solution of 3,5-dibromo-4-[2-(1-hydroxyethyl)-5-isopropyl-4-methoxyphenoxy]-phenylacetic acid methyl ester (40 mg, 0.08 mmol) in CH₂Cl₂ (3 mL). The mixture was stirred at room temperature for 16 hours, poured into water, and extracted twice with AcOEt.

The combined organic extracts were washed with brine, dried with Mg₂SO₄ and concentrated. The residue was purified by chromatography to give the title compound (12 mg, 25%). ¹H NMR (CDCl₃): δ 8.0 (broad s, 1H), 7.7-7.6 (m, 3H), 7.4-7.1 (m, 3H), 7.0 (appt, 1H), 6.6 (s, 1H), 6.1 (s, 1H), 5.0 (q, 1H), 3.7 (s, 2H), 3.5 (s, 3H), 1.78 (d, 3H), 1.0 (m, 6H)

### EXAMPLE 3

4-[2-(2-Cyclopentyl-1-hydroxyethyl)-5-isopropyl-4-methoxyghenoxy]-3,5-dibromophenylaceti c Acid (E3).

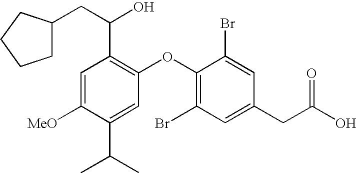

Step 1.

4-(2-Cyclopentylacetyl-5-isopropyl-4-methoxyphenoxy)-3,5-dibromophenylacetic Acid Methyl Ester.

The compound was prepared by the method described in Example 1, Step 3, from 3,5-dibromo-4-(3-isopropy-4-methoxyphenoxy)phenylacetic acid methyl ester (400 mg, 0.84 mmol) and cyclopentylacetyl chloride (460 mg, 3.14 mmol) to give a material (765 mg) that was used in the next step without further purification.

Step 2.

4(2-Cyclopenbdacetyl-5-isopropyl-4-methoxyphenoxy)-3,5-dibromophenylacetic Acid.

1M LiOH (1.5 mL) was added to a solution of 4-(2-cyclopentylacetyl-5-isopropyl-4-methoxyphenoxy)-3,5-dibromophenylacetic acid methyl ester (380 mg, 0.65 mmol) in THF (1.5 mL). The mixture was stirred at room temperature overnight, acidified with 2M HCl and extracted twice with ethylacetate. The combined organic phases were washed with water and brine, dried over Na₂SO₄ and concentrated. The residue was dried under vacuum to give 91 mg (25%) of the title compound as a white solid. ¹H NMR (CDCl₃): δ 7.56 (s, 2H), 7.34 (s, 1H), 6.19 (s, 1H), 3.83 (s, 3H), 3.64 (s, 2H), 3.30-3.10 (m, 3H), 2.55-2.40 (m, 1H), 1.90-1.75 (m, 2H), 1.70-1.45 (m, 4H), 1.30-1.00 (m, 2H),1.03 (d, 6H).

Step 3.

4-[2-(2-Cyclopentyl-1-hydroxyethyl)-5-isopropyl-4-methoxyphenoxy]-3,5-dibromophenylacetic Acid.

The compound was obtained as a white solid by the method described in Example 1, Step 4, from 4-(2-cyclopentylacetyl-5-isopropyl-4-methoxyphenoxy)-3,5-dibromophenylacetic acid (45 mg, 0.08 mmol) and NaBH₄. Yield: 26 mg (56%). ¹H NMR (MeOD): δ 7.65 (d, 2H), 7.11 (s, 1H), 6.07 (s, 1H), 5.35-5.25 (m, 1H), 3.80 (s, 3H), 3.67 (s, 2H), 3.40-3.10 (m, 3H), 2.25-2.00 (m, 1H), 2.00-1.7 (m, 2H), 1.70-1.40 (m, 4H), 1.40-1.10 (m, 2H), 1.10-0.90 (m, 6H).

### Example 4

### 3,5-Dibromo-4-{[2-(hydroxy(phenyl)methyl)]-5-isopropyl-4-methoxyphenoxy}-phenylacetic Acid (E4)

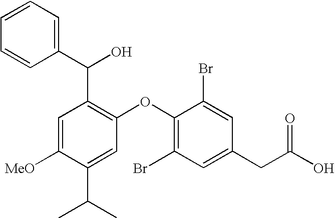

Step 1.

4(2-Benzoyl-5-isopropyl-4-methoxyphenoxy)-3,5-dibromophenylacetic Acid Methyl Ester.

TiCl₄ (13.9 mL, 127 mmol) was added dropwise to a solution of 3,5-dibromo-4-(3-isopropyl-4-methoxyphenoxy)phenylacetic acid methyl ester (20.0 g, 42.3 mmol) and benzoylchloride (17.8 g, 127 mmol) in CH₂Cl₂ (250 mL) at 0° C. The mixture was stirred at room temperature for 5 d, cooled to 0° C. and quenched carefully with water (50 mL). NaHCO₃ (aq., sat, 100 mL) was added, the mixture stirred at room temperature for 1 h and extracted with EtOAc (3×100 mL). The combined extracts were concentrated and purified by chromatography on silica gel to give 15.2 g (62%) of the title compound. ¹H NMR (CDCl₃): δ 8.01 (m, 3H), 7.58-7.38 (m, 4H), 7.00 (s, 1H), 6.26 (s, 1H), 3.82 (s, 3H), 3.69 (s, 3H), 3.54 (s, 2H), 3.24 (m, 1H), 1.09 (d, 6H).

Step 2.

3,5-Dibromo-4-{[2-(hydroxy-(phenyl)methyl)]-5-isopropyl-4-methoxyphenoxy}phenylacetic Acid.

The compound was prepared as described in Example 1, Step 4. ¹H NMR (CDCl₃): δ 7. 7.6-7.5 (m, 4H), 7.3-7.2 (m, 3H), 6.9 (s, 1H), 6.4 (s, 1H), 6.1 (s, 1H), 3.8 (s, 3H), 3.6 (s, 2H), 3.2 (m, 1H), 1.0 (d, 6H).

### EXAMPLE 5

### 3,5-Dibromo-4-{5-isopropyl-4-methoxy-2-[(2-{methylsulfonyl)ethoxy(phenyl)-methyl]phenoxy}phenylacetic Acid (E5)

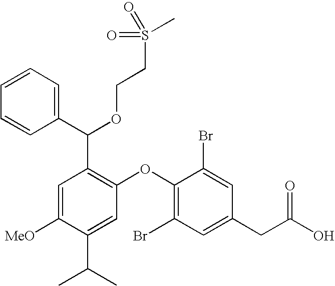

Step 1.

3,5-Dibromo-4-{5-isopropyl-4-methoxy-2-[(2-methylsulfonyl)ethoxy(phenyl)methyl]-phenoxy)phenylacetic Acid (methylsulfonyl)ethyl Ester.

Concentrated H₂SO₄ (0.02 mL) was added to a stirred solution of 3,5-dibromo-4-{[2-(hydroxy-(phenyl)methyl)]-5-isopropyl-4-methoxyphenoxy}phenylacetic acid (58 mg, 0.1 mmol), prepared according to Example 4, Step 2, in (methylsulfonyl)-ethanol (1 mL) at room temperature. The solution was stirred for 17 h at 70° C., diluted with EtOAc, washed with NaHCO₃ (aq., sat), water and brine, dried over MgSO₄ and concentrated. The oily residue was purified by chromatography on silica gel to give 25 mg (31%) of the title compound. ¹H NMR (CDCl₃): δ 7.5-7.4 (m, 4H), 7.3-7.2 (m, 3H), 6.9 (s, 1H), 6.11 (s, 1H), 6.09 (s, 1H), 4.654.55 (m, 2H), 4.15-3.95 (m, 2H), 3.8 (s, 3H), 3.7 (s, 2H), 3.4-3.3 (m, 4H), 3.2-3.1 (m, 1H), 3.0 (s, 3H), 2.9 (s, 3H), 1.1-0.9 (m, 6H).

Step 2.

3,5-Dibromo-4-{5-isopropyl-4-methoxy-2-[(2-methylsulfonyl)ethoxy(phenyl)methyl]-phenoxy}phenylacetic Acid.

The compound was prepared as described in Example 3, step 2. ¹H NMR (CDCl₃): δ 7.5-7.4 (m, 4H), 7.3-7.2 (m, 3H), 6.9 (s, 1H), 6.11 (s, 1H), 6.09 (s, 1H), 4.15-3.95 (m, 2H), 3.8 (s, 3H), 3.7 (s, 2H), 3.4-3.3 (m, 1H), 3.2-3.1 (m, 1H), 3.0-2.9 (m, 5H), 1.1-0.9 (m, 6H).

### EXAMPLE 6

### 3,5-Dibromo-4-{2-[hydroxy(2-methylphenyl)methyl]-5-isopronyl-4-methoxyphenoxy}phenylacetic Acid (E6)

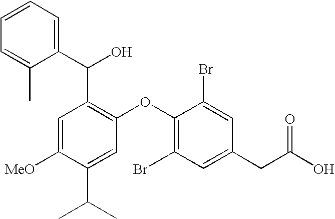

Step 1.

3,5-Dibromo-4-[5-isopropyl-4-methoxy-2-(2-methylbenzoyl)phenoxy]phenylacetic Acid Methyl Ester.

The compound was obtained as a white solid by the method described in Example 1, Step 3, from 3,5-dibromo-4-(3-isopropyl-4-methoxyphenoxy)phenylacetic acid methyl ester (20.0 g, 42.3 mmol) and o-toluoyl chloride (22.9 g, 154.0 mmol). Yield: 12.7 g (54%).

Step 2.

3,5-Dibromo-4-{2-[hydroxy(2-methylphenyl)methyl]-5-isopropyl-4-methoxyphenoxy}-phenylacetic Acid.

The compound was obtained by the method described in Example 1, Step 4, from 3,5-dibromo-4-[5-isopropyl-2-(2-methylbenzoyl)₄-methoxyphenoxy]phenylacetic acid methyl ester (7.9 g, 13.4 mmol). Yield: 5.4 g (70%). ¹H NMR (DMSO-d₆): δ 12.20 (s, 1H), 7.65 (m, 2H), 7.24 (m, 2H), 7.11 (s, 2H), 6.32 (s, 1H), 5.97 (s, 1H), 5.66 (s, 1H), 3.78 (s, 3H), 3.66 (s, 2H), 3.11 (m, 1H), 2.42 (s, 3H), 0.94 (dd, 6H).

### EXAMPLE 7

3,5-Dibromo-4-{2-[(2,4-difluorophenoxy)(2-methylphenyl)methyl]-5-isopropyl-4-methoxyphenoxy}phenylacetic Acid (E7).

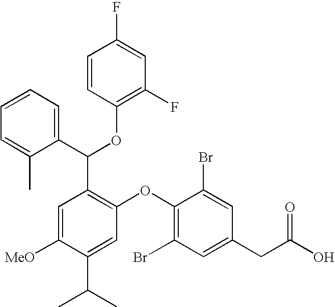

Step 1.

3,5-Dibromo-4-{5-isopropyl-4-methoxy-2-[methoxy(2-methylphenyl)methyl]phenoxy}-phenylacetic Acid Methyl Ester.

Thionyl chloride (3 drops) was added to a solution of 3,5-dibromo-4-{2-[hydroxy-(2-methylphenyl)methyl]-5-isopropyl-4-methoxy-phenoxy}phenylacetic acid (1.0 g, 1.73 mmol), prepared as described in Example 6, Step 2, in MeOH (3 mL). The solution was stirred at room temperature for 12 and concentrated. The residue was used without further purification in the next step.

Step 2.

4-{2-[Chloro(2-methylphenyl)methyl]-5-isopropyl-4-methoxyphenoxy}3,5-dibromophenylacetic Acid Metyl Ester.

Thionyl chloride (25 μL, 0.34 mmol) was added to a solution of 3,5-dibromo-4-{2-[hydroxy(2-methylphenyl)methyl]-5-isopropyl-4-methoxyphenoxy}phenylac etic acid methyl ester (100 mg, 0.17 mmol) in anhydrous CH₂Cl₂ (3 mL). The solution was stirred at room temperature for 12 and concentrated. The residue was used without further purification in the next step.

Step 3.

3,5-Dibromo-4-{2-[(2,4-difluorophenoxy)(2-methylphenyl)methyl]-5-isopropyl-4-methoxyphen oxy}phenylacetic Acid Methyl Ester.

The 4-{2-[chloro(2-methylphenyl)methyl]-5-isopropyl-4-methoxyphenoxy}-3,5-dibromophenylacetic acid metyl ester prepared as above was dissolved in MeCN (2 mL). 2,4-Difluorophenol (0.11 g, 0.85 mmol) and N-ethyldiisopropylamine (0.15 mL, 0.85 mmol) was added and the mixture was stirred at 60° C. for 12 h. The mixture was concentrated and the residue was used without further purificafion in the next step.

Step 4.

3,5-Dibromo-4-{2-[(2,4-dinuorophenoxy)(2-methylphenyl)methy]-5-isopropyl-4-methoxyphenoxy}phenylacetic Acid.

The compound from Step 3 was dissolved in MeOH/aq. 1 M. NaOH (3:1, 2 mL) and the mixture was heated at 40° C. for 12 h, allowed to cool to room temperature, acidified with 1 M HCl and extracted twice with EtOAc. The combined extracts were washed twice with water, concentrated and purified by HPLC to give the title product. ¹H NMR (CDCl₃): δ 7.5-7.4 (m, 3H), 7.2-7.1 (m, 3H), 7.0-6.9 (m, 3H), 6.8-6.7 (m, 1H), 6.1 (s, 11H), 3.7 (s, 3H), 3.4 (s, 2H), 3.1 (m, 1H), 2.4 (s, 3H), 1.1-0.9 (m, 6H).

### EXAMPLE 8

### 3,5-Dibromo-4-{2-[2-butylamino(2-methylphenyl)methyl]-5-isopropyl-4-methoxyphenoxy}phenylatetic Acid (E8)

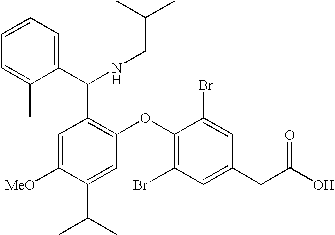

Step 1.

3,5-Dibromo-4-{2-[2-butylamino(2-methylphenyl)methyl-]5-isopropyl-4-methoxy-phenoxy}ph enylacetic acid methyl ester.

3,5-Dibromo-4-{2-[chloro(2-methylphenyl)methyl]-5-isopropyl-4-methoxyphenoxy}-phenylacetic acid metyl ester was prepared as described in Example 7, Step 2, and dissolved in MeCN (2 mL). Isobutylamine (85 μL, 0.85 mmol) was added and the mixture was stirred at 60° C. for 12 h, allowed to cool to room temperature and concentrated. The residue was used without further purification in the next step.

Step 2.

3,5-Dibromo-4-{2-[2-butylamino(2-methylphenyl)methyl]-5-isopropyl-4-methoxyphenoxy}phenylacetic Acid.

The compound was obtained as described in Example 7, Step 4. ¹H NMR (CDCl₃): δ 7.5-7.1 (m, 6H), 6.9 (s, 1H), 6.2 (s, 1H), 5.9 (s, 1H), 3.7 (s, 3H), 3.5 (s, 2H), 3.1 (m, 1H), 2.7 (m, 2H), 2.4 (s, 3H), 2.1-1.9 (m, 1H), 1.1-0.9 (m, 12H).

### EXAMPLE 9

3,5-Dibromo-4-}2-[hydroxy(3-methylphenyl)methyl]-5-isopropyl-4-methoxy-phenoxy}phenyla cetic Acid (E9).

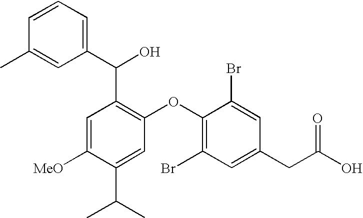

Step 1.

3,5-Dibromo-4-[5-isopropyl-4-methoxy-2-(3-methylbenzoyl)phenoxy]phenylacetic Acid Methyl Ester.

The compound was prepared as described in Example 1, Step 3, from 3,5-dibromo-4-(3-isopropyl-4-methoxyphenoxy)phenylacetic acid methyl ester (50 mg, 0.11 mmol) and 3-methylbenzoyl chloride (82 mg, 0.53 mmol). Yield: 52 mg (83%).

Step 2.

3,5-Dibromo-4-[5-isopropyl-4-methoxy-2-(3-methylbenzoyl)phenoxy]phenylactic acid. 3,5-Dibromo-4-[5-isopropyl-4-methoxy-2-(3-methylbenzoyl)phenoxy]phenylacetic acid methyl ester (25 mg, 0.04 mmol) was dissolved in a 3:5 mixture of 1 M NaOH/MeOH (2 mL). The mixture was stirred at room temperature for 17 h, acidified with 1 M HCl, concentrated to a small volume and extracted twice with EtOAc. The combined organic phases were dried over MgSO₄, concentrated and the residue dried under vacuum to give 19 mg (80%) of the title compound. ¹H NMR (CDCl₃): δ 7.80 (m, 2H), 7.42 (s, 2H), 7.33 (m, 2H), 6.98 (s, 1H), 6.24 (s, 1H), 3.80 (s, 3H), 3.57 (s, 2H), 3.22 (m, 1H), 2.37 (s, 3H), 1.07 (d, 6H).

Step 3.

3,5-Dibromo-4-[2-(hydroxy(3-methylphenyl)methyl)-5-isopropyl-4-methoxyphenoxy]-phenylacetic Acid.

LiOH (1M) was added dropwise to a suspension of 3,5-dibromo-4-[5-isopropyl-4-methoxy-2-(3-methylbenzoyl)phenoxy]phenylacetic acid (52 mg, 0.09 mmol) in water (2.5 mL) until the acid had dissolved. NaBH₄ (11 mg, 0.29 mmol) was added in one portion at room temperature. After 40 h the solution was acidified with 2M HCl and extracted twice with EtOAc. The combined extracts were washed with water and brine, and dried over MgSO₄. Concentration gave the title compound (40 mg, 77%).

¹H NMR (CDCl₃): δ 7.5 (s, 2H), 7.3-7.2 (m, 3H), 7.0 (m, 1H), 6.9 (s, 1H), 6.3 (s, 1H), 6.1 (s, 1H), 3.7 (s, 3H), 3.6 (s, 2H), 3.2 (m, 1H), 2.3 (s, 3H), 1.0 (d, 6H).

### EXAMPLE 10

### 3,5-Dibromo-4-[5-isopropyl-4-methoxy-2-(methoxy(3-methylphenyl)methyl)-phenoxy]phenylacetic Acid (E10)

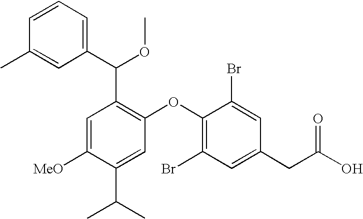

Step 1.

3,5-Dibromo-4-[5-isopropyl-4-methoxy-2-(methoxy(3-methylphenyl)methyl)-phenoxy]phenylacetic Acid Methyl Ester.

Concentrated H₂SO₄ (0.1 mL) was added to a stirred solution of 3,5-dibromo-4-{2-[hydroxy(3-methylphenyl)-methyl]-5-isopropyl-4-methoxyphenoxy}phenylac etic acid (100 mg, 15 mmol), prepared as described in Example 9, in metanol (5 mL) at room temperature. After 17 h at 70° C. the mixture was allowed to cool to room temperature. Filtration gave the title compound (67 mg, 65%).

Step 2.

3,5-Dibromo-4-[5-isopropyl-4-methoxy-2-(methoxy(3-methylphenyl)methyl)-phenoxy]phenylacetic Acid.

The compound was obtained as described in Example 3, Step 2. ¹H NMR (CDCl₃): δ 7.6 (s, 2H), 7.4 (s, 1H), 7,2 (m, 2H), 7,0 (m, 2H), 6,1 (s, 1H), 5.9 (s, 1H), 3.8 (s, 3H), 3.7 (s, 2H), 3.5 (s, 3H), 3.14 (m, 1H), 2.3 (s, 3H), 1.0 (m, 6H).

### EXAMPLE 11

### 3,5-Dibromo-4-{2-[isoprooxy(3-methylphenyl)methyl]-5-isopropyl-4-methoxyphenoxy}phenylacetic Acid (E11)

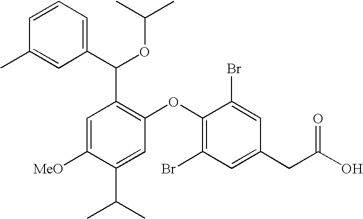

Step 1.

3,5-Dibromo-4-{2-[isopropoxy(3-mothylphenyl)methyl]-5-isopropyl-4-methoxyphenoxy}phenylacetic Acid Isopropyl Ester.

Concentrated H₂SO₄ (0.01 mL) was added to a stirred solution of 3,5-dibromo-4-{2-[hydroxy-(3-methylphenyl)-methyl]-5-isopropyl-4-methoxyphenoxy}phenyla cetic acid (72 mg), prepared as described in Example 9, in 2-propanol (3 mL) at room temperature. After 17 h at 70° C., the solution was concentrated and the residue dissolved in EtOAc. The solution was washed with water and brine and dried over MgSO₄. Concentration gave 40 mg (50%) of the title compound as a white solid.

Step 2.

3,5-Dibromo-4-{2-[isopropoxy(3-methylphenyl)methyl]-5 isopropyl/methoxyphenoxy}phenylacetic Acid.

The compound was obtained as described in Example 3, Step 2. ¹H NMR (CDCl₃): δ 7.5 (s, 2H), 7.4 (m, 2H), 7,2 (t, 1H), 7,0 (m, 2H), 6,2 (s, 1H), 6.0 (s, 1H), 3.8 (m, 4H), 3.6 (s, 2H), 3.1 (m, 1H), 2.3 (s, 3H), 1.3 (m, 6H), 1.0 (m, 6H).

### EXAMPLE 12

4-{2-[Cyclohexyloxy(3-methylphenylmethyl]-5-isopropyl-4-methoxyphenoxy}-3,5-dibromoph enylacetic Acid E12).

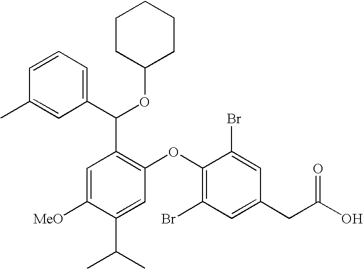

Step 1.

4-{2-[Cyclohexyloxy(3-methylphenyl)methyl-5-isopropyl-4-methoxyphenoxy}-3,5-dibromoph enylacetic Acid Cyclohexyl Ester.

Concentrated H₂SO₄ (0.02 mL) was added to a stirred solution of 3,5-dibromo-4-{2-[hydroxy-(3-methylphenyl)-methyl]-5-isopropyl-4-methoxyphenoxy}phenyla cetic acid (206 mg, 0.36 mmol), prepared as described in Example 10, in cyclohexanol (4 mL) at room temperature. The solution was stirred for 17 h at 70° C., diluted with EtOAc, washed with NaHCO₃ (aq., sat), water and brine, dried over MgSO₄ and concentrated. The oily residue was purified by chromatography on silica gel to give 159 mg (62%) of the title compound.

Step 2.

4(2-[Cyclohexyloxy(3-methylphenyl)methyl]-5-isopropyl-4-methoxyphenoxy}-3,5-dibromoph enylacetic Acid.

The compound was obtained as described in Example 3, Step 2. ¹H NMR (CDCl₃): δ 7.5 (s, 2H), 7.4 (m, 2H), 7,2 (t, 1H), 7,0 (m, 2H), 6,2 (s, 1H), 6.0 (s, 1H), 3.7 (s, 3H), 3.6 (s, 2H), 3.4 (m, 1H), 3.1 (m, 1H), 2.3 (s, 3H), 2.2-1.9 (m, 2H), 1.8-1.7 (m, 2H), 1.5-1.4 (m, 3H), 1.3-1.1 (3H), 1.0 (m, 6H).

### EXAMPLE 13

3,5-Dibromo-4-{2-[(4-fluorophenoxy)(3-methylphenyl)methyl]-5-isopropyl-4-methoxyphenoxy}phenylacetic Acid (E13).

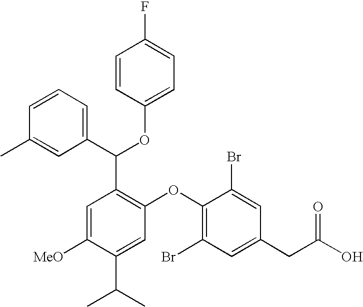

4-Fluorophenol (112 mg, 1 mmol) followed by tin(II)chloride dihydrate (250 mg, 1 mmol) was added to a solution of 3,5-Dibromo-4-{2-[hydroxy(3-methylphenyl)-methyl]-5-isopropyl-4-methoxyphenoxy}phenylac etic acid (65 mg, 0.11 mmol), prepared as described in Example 9, in CH₂Cl₂ (5 mL). The mixture was stirred at room temperature for two days, poured into water, and extracted twice with AcOEt. The combined organic extracts were washed with brine, dried with Mg₂SO₄ and concentrated. The residue was purified by chromatography on silica gel to give 13.9 mg (19%) of the title compound. ¹H NMR (CDCl₃): δ 7.6 (s, 2H), 7.4-6.8 (m, 9H), 6.7 (s, 1H), 6.1 (s, 1H), 3.7 (s, 3H), 3.6 (s, 2H), 3.2 (m, 1H), 2.3 (s, 3H), 1.0 (d, 6H).

### EXAMPLE 14

### 3,5-Dibromo-4-{5-isopropyl-4-methoxy-2-[(4-methoxyphenoxy)(3-methylphenyl)methyl]phenoxy}phenylacetic Acid (E14)

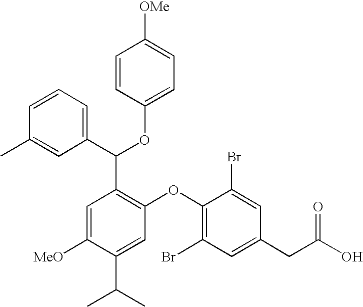

4-Methoxyphenol (838 mg, 6.7 mmol) followed by tin(II)chloride dihydrate (188 mg, 0.87 mmol) was added to a solution of 3,5-dibromo-4-{2-[hydroxy-(3-methylphenyl)-methyl]-5-isopropyl-4-methoxyphenoxy}phenylacetic acid (390 mg, 0.67 mmol), prepared as described in Example 9, in CH₂Cl₂ (5 mL). The mixture was stirred at room temperature for 20 hours, poured into water, and extracted twice with AcOEt. The combined extracts were washed with brine, dried with Mg₂SO₄ and concentrated. The residue was purified by HPLC to give the title compound (35 mg, 8%). ¹H NMR (CDCl₃): δ 7.6 (s, 2H), 7.5 (m, 2H), 7.3-7.1 (m, 7H), 6.1 (s, 1H), 3.7 (s, 6H), 3.6 (s, 2H), 3.2 (m, 1H), 2.3 (s, 3H), 1.0 (d, 6H).

### EXAMPLE 15

### 3,5-Dibromo-4-{5-isopropyl-4-methoxy-2-[(3-methylphenyl)(4-nitrophenoxy)-methyl]phenoxy}phenylacetic Acid (E15)

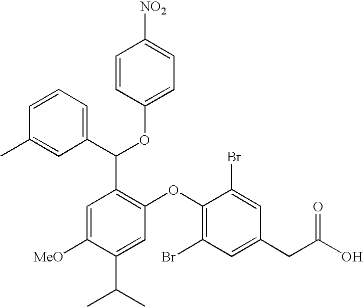

4-Nitrophenol (556 mg, 4 mmol) followed by tin(II)chloride dihydrate (135 mg, 0.6 mmol) was added to a solution of 3,5-Dibromo-4-{2-[hydroxy-(3-methylphenyl)-methyl]-5-isopropyl-4-methoxyphenoxy}phenylacetic acid (130 mg, 0.4 mmol), prepared as described in Example 9, in CH₂Cl₂ (10 mL). The mixture was stirred at room temperature for 16 hours, poured into water, and extracted twice with AcOEt. The combined organic extracts were washed with brine, dried with Mg₂SO₄ and concentrated. The residue was purified by chromatography on silica gel to give the title compound (42 mg, 15%). ¹H NMR (CDCl₃): δ 8.1 (d, 2H), 7.5 (m, 2H), 7.2-7.0 (m, 6H), 6.9 (d, 2H), 6.1 (s, 1H), 3.7 (2s, 5H), 3.2 (m, 1H), 2.3 (s, 3H), 1.0 (d, 6H).

### EXAMPLE 16

3,5-Dibromo-4-{2-[(4-aminophenoxy)(3-methylphenyl)methyl]-5-isopropyl-4-methoxyphenox y}phenylacetic Acid (E16).

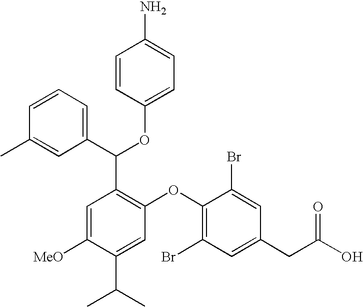

A mixture of 3,5-Dibromo-4-{5-isopropyl-4-methoxy-2-[(4-nitrophenoxy)(3-methylphenyl)methyl]-phenoxy}phenylacetic acid (24 mg, 0.034 mol), prepared as described in Example 15, Na₂S₂O₄ (70 mg, 0.4 mmol) and ethanol (5 mL) was heated under reflux for 16 h. The reaction mixture was cooled to room temperature, filtered, concentrated, and purified by HPLC to give 7 mg (31%) of the title compound. ¹H NMR (CD₃OD): δ 7.7 (d, 2H), 7.3 (m, 2H), 7.2-7.0 (m, 7H), 6.9 (s, 1H), 6.1 (s, 1H), 3.7 (2s, 5H), 3.2 (m, 1H), 2.3 (s, 3H), 1.0 (d, 6H).

### EXAMPLE 17

3,5-Dibromo-4-{2-[(4-hydroxybenzoyloxy)(3-methylphenyl)methyl]-5-isopropyl-4-methoxy]ph enoxy}phenylacetic Acid (E17).

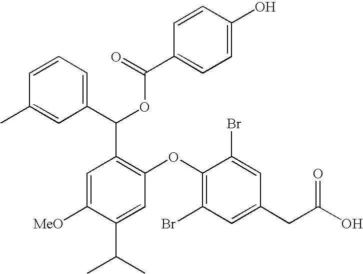

4-Hydroxybenzoic acid (276 mg, 2 mmol) followed by tin(II)chloride dihydrate (67 mg, 0.3 mmol) was added to a solution of 3,5-dibromo-4-{2-[hydroxy(3-methylphenyl)-methyl]-5-isopropyl-4-methoxyphenoxy}phenylacetic acid (115 mg, 0.2 mmol), prepared as described in Example 10, in CH₃CN (5 mL). The mixture was stirred at room temperature for 16 hours, poured into water, and extracted twice with AcOEt. The combined organic extracts were washed with brine, dried with Mg₂SO₄ and concentrated. The residue was purified by preparative HPLC to give the title compound (12 mg, 9%). ¹H NMR (CDCl₃): δ 8.0 (d, 2H), 7.5-6.8 (m, 10H), 6.1 (s, 1H), 3.7 (2s, 5H), 3.1 (m, 1H), 2.3 (s, 3H), 1.0 (d, 6H).

### EXAMPLE 18

### 4-}2-[Chloro(3-methylphenyl)methyl]-5-isopropyl-4-methoxyphenoxy}-3,5-dibromophenylacetic Acid (E18)

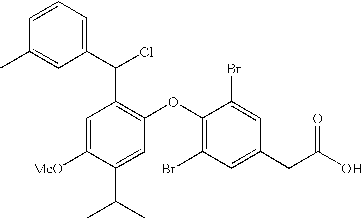

A mixture of 3,5-dibromo-4-{2-[hydroxy(3-methylphenyl)methyl]-5-isopropyl-4-methoxyphenoxy}phenylacetic acid (1.9 g, 3.3 mmol), prepared as described in Example 9, concentrated HCl (10 mL) and chloroform (10 mL) was stirred vigorously for 1d. The organic layer was separated and washed with water, brine and concentrated to give the title compound (1.9 g, 96%). ¹H NMR (CDCl₃): δ 7.5-7.1 (m, 7H), 6.7 (s, 1H), 6.1 (s, 1H), 3.8 (s, 3H), 3.6 (s, 2H), 3.2 (m, 1H), 2.3 (s, 3H), 1.0 (m, 6H).

### EXAMPLE 19

### 4-{2-[Amino(3-methylphenyl)methyl]-5-isopropyl-4-methoxyphenoxy}-3,5-dibromophenylacetic Acid (E19)

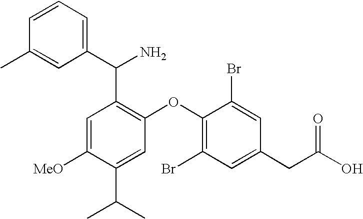

A mixture of 3,5-dibromo-4-{2-[chloro(3-methylphenyl)methyl]-5-isopropyl-4-methoxyphenoxy}phenylacetic acid (596 mg, 1 mmol), prepared as described in Example 18, and concentrated aqueous ammonia (20 mL) was heated in a sealed tube at 50° C. for 1 d. The reaction was cooled to room temperature and was extracted with AcOEt. The extract was concentrated and the residue purified by chromatography to give the title compound (156 mg, 27%). ¹H NMR (CD₃OD): δ, 7.6 (s, 2H), 7.3-7.0 (m, 5H), 6.2 (s, 1H), 5.8 (s, 1H), 3.8 (s, 3H), 3.5 (s, 2H), 2.3 (s, 3H), 1.0 (d, 6H).

### EXAMPLE 20

3,5-Dibromo-4-{2-[isopropylamino(3-methylphenyl)methyl]-5-isopropyl-4-methoxyphenoxy}phenylacetic Acid (E20).

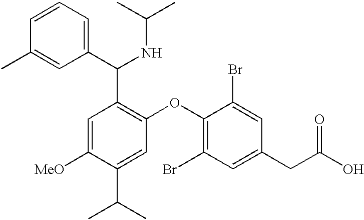

Step 1.

3,5-Dibromo-4-{2-[isopropylamino(3-methylphenyl)methyl]-5-isopropyl-4-methoxyphenoxy}phenylacetic Acid Methyl Ester.

The compound was prepared according to the method described in Example 8 from 4-{2-[chloro(3-methylphenyl)methyl]-5-isopropyl-4-methoxyphenoxy}3,5-dibromo-phenylacetic acid metyl ester, prepared as described in Example 7, Step 2, from thionyl chloride and 3,5-dibromo-4-{2-[hydroxy(3-methylphenyl)-methyl]-5-isopropyl-4-methoxyphenoxy}-phenylacetic acid, prepared as described in Example 9, and isopropylamine. The material was used without further purification in the next step.

Step 2.

3,5-Dibromo-4-{2-[isopropylamino(3-methylphenyl)methyl]-5-isopropyl-4-methoxyphenoxy}phenylacetic Acid.

The compound was obtained as described in Example 7, Step 4. ¹H NMR (CDCl₃): δ 7.8-7.0 (m, 7H), 6.2 (s, 1H), 6.0 (s, 1H), 3.9 (s, 3H), 3.5 (s, 2H), 3.4-3.3 (m, 1H), 3.2-3.1 (m, 1H), 2.3 (s, 3H), 1.5-1.4 (m, 6H), 1.10.9 (m, 6H).

### EXAMPLE 21

### 4-{2-[Cyclopropylamino(3-methylphenyl)methyl]-5-isopropyl-4-methoxyphenoxy}-3,5-dibromo-phenylacetic Acid (E21)

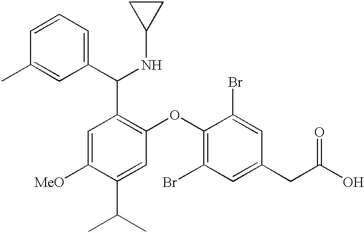

The title compound was prepared according to the procedure described in Example 20 from cyclopropylamine. ¹H NMR (CDCl₃): δ 7.5-7.4 (m, 2H), 7.3-7.1 (m, 3H), 7.1-7.0 (m, 2H), 6.2 (s, 1H), 5.6 (s, 1H), 3.8 (s, 3H), 3.5 (s, 2H), 3.1 (m, 1H), 2.42.2 (m, 4H), 1.1-0.9 (m, 6H), 0.7-0.6 (m, 2H), 0.5-0.4 (m, 2H)

### EXAMPLE 22

3,5-Dibromo-4-{2-[(1-pyrrolidino(3-methylphenyl)methyl]-5-isopropyl-4-methoxyphenoxy}phenylacetic Acid (E22).

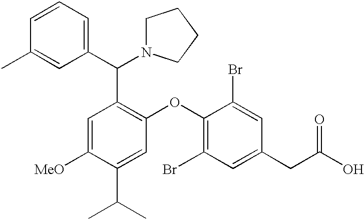

The title compound was prepared according to the procedure described in Example 20 from pyrrolidine. ¹H NMR (CDCl₃): δ 7.8-7.5 (m, 5H), 7.3-7.2 (m, 1H), 7.1-7.0 (m, 1H), 6.1 (s, 1H), 5.6 (s, 1H), 3.7 (s, 3H), 3.6 (s, 2H), 3.43.2 (m, 4H), 3.1 (m, 1H), 2.3 (s, 3H), 2.2-2.0 (m, 4H), 1.0-0.8 (m, 6H).

### EXAMPLE 23

3,5-Dibromo-4-{2-[(1-piperidino)(3-methylphenyl)methyl]-5-isopropyl-4-methoxyphenoxy}phenylacetic Acid (E23).

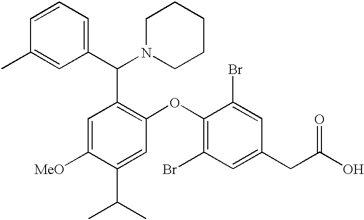

The title compound was prepared according to the procedure described in Example 20 from piperidine. ¹H NMR (CDCl₃): δ 7.8-7.4 (m, 5H), 7.3-7.2 (m, ₁H), 7.1-7.0 (m, 1H), 6.0 (s, 1H), 5.6 (s, 1H), 3.7 (s, 3H), 3.6 (s, 2H), 3.2-2.9 (m, 5H), 2.3 (s, 3H), 1.9-1.7 (m, 4H), 1.6-1.4 (m,2H), 1.0-0.7 (m, 6H).

### EXAMPLE 24

3,5-Dibromo-4-{2-[(2-methoxy-1-ethyl)amino(3-methylphenyl)methyl]-5-isopropyl-4-methoxyphenoxy}phenylacetic Acid (E24).

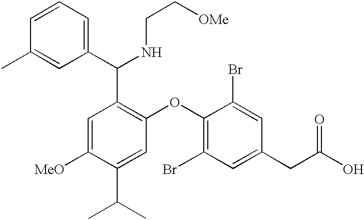

The title compound was prepared according to the procedure described in Example 20 from 2-methoxyethylamine. ¹H NMR (CDCl₃): δ 7.5-7.0 (m, 7H), 6.2 (s, 1H), 5.7 (s, 1H), 3.8 (s, 3H), 3.6 (m, 2H), 3.5 (s, 2H), 3.3 (s, 3H), 3.2-3.0 (m, 3H), 2.3 (s, 3H), 1.0 (d, 6H).

### EXAMPLE 25

### 3,5-Dibromo-4-{2-[(2-{N,N-diethylaminol}-1-ethyl)amino(3-methylphenyl)-methyl]-5-isopropyl-4-methoxyphenoxy}phenylacetic Acid (E25)

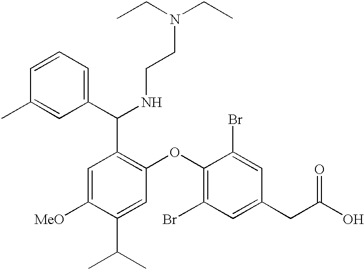

The title compound was prepared according to the procedure described in Example 20 from N,N-diethylethylenediamine. ¹H NMR (CDCl₃): δ 7.5-7.1 (m, 6H), 6.9 (s, 1H), 6.2 (s, 1H), 5.4 (s, 1H), 3.8 (s, 3H), 3.4 (s, 2H), 3.3-2.9 (m, 9H), 2.3 (s, 3H), 1.2 (t, 6H), 1.1-0.9 (m, 6H).

### EXAMPLE 26

### 3,5-Dibromo-4-{2-[(3-methylphenyl)(2-{1-piperidino}-1-ethyl)amino-methyl]-5-isopropyl-4-methoxyphenoxy}phenylacetic Acid (E26)

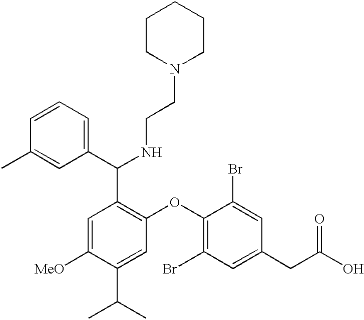

The title compound was prepared according to the procedure described in Example 20 from 1-(2-aminoethyl)piperidine. ¹H NMR-(CDCl₃): δ 7.4-7.0 (m, 6H); 6.9 (s, 1H), 6.3 (s, 1H), 5.4. (s, 1H), 3.8 (s, 3H), 3.4 (s, 2H), 3.2-2.8 (m, 9H), 2.3 (s, 3H), 1.9-1.7 (m, 4H), 1.6-1.4 (m, 2H), 1.0 (d, 6H).

### EXAMPLE 27

3,5-Dibromo-4-{2-[(1-piperazino)(3-methylphenyl)methyl]-5-isopropyl-4-methoxyphenoxy}phenylacetic Acid (E27).

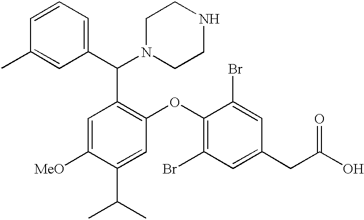

The title compound was prepared according to the procedure described in Example 20 from piperazine. ¹H NMR (CDCl₃): δ 7.7-6.9 (m, 7H), 6.1 (s, 1H), 5.1 (s, 1H), 3.7 (s, 3H), 3.5 (s, 2H), 3.33.0 (m, 5H), 2.8-2.5 (m, 4H), 2.3 (s, 3H), 1.1-0.9 (m, 6H).

### EXAMPLE 28

### 3,5-Dibromo-4-{2-[4-methoxybenzylamino(3-methylphenyl)methyl]-5-isopropyl-4-methoxyphenoxy}phenylacetic Acid (E28)

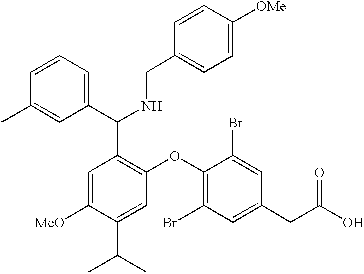

The title compound was prepared according to the procedure described in Example 20 from 4-methoxybenzylamine. ¹H NMR (CDCl₃): δ 7.5-7.0 (m, 9H), 6.8 (m, 2H), 6.2 (s, 1H), 5.7 (s, 1H), 4.1 (d, 1H), 4.9 (d, 1H), 3.8 (s, 3H), 3.7 (s, 3H), 3.4 (s, 2H), 3.1 (m, 1H), 2.3 (s, 3H), 1.1-0.9 (m, 6H).

### EXAMPLE 29

### 4-{2-[(3-Carboxyphenyl)amino(3-methylphenyl)methyl]-5-isopropyl-4-methoxyphenoxy}-3,5-dibromo-phenylacetic Acid (E29)

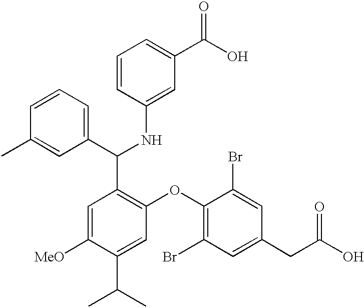

The title compound was prepared according to the procedure described in Example 20 from ethyl 3-aminobenzoate. ¹H NMR (CDCl₃): δ 7.5-6.7 (m, 11H), 6.1 (s, 1H), 6.0 (s, 1H), 3.6 (s, 3H), 3.4 (s, 2H), 3.1 (m, 1H), 2.3 (s, 3H), 1.1-0.9 (m, 6H).

### EXAMPLE 30

3,5-Dibromod-4-{2-[(N-{4-hydrozybenzoyl}amino)(3-methylphenyl)methyl]-5-isopropyl-4-meth oxyphenoxy}phenylacetic Acid (E30).

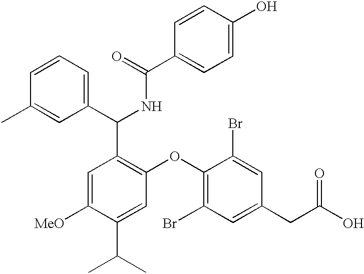

The title compound was prepared as described in Example 17 from 4-hydroxybenzamide (274 mg, 2 mmol) and 3,5-Dibromo-4-{2-[hydroxy-(3-methylphenyl)-methyl]-5-isopropyl-4-methoxyphenoxy}phenyla cetic acid (180 mg, 0.31 mmol), prepared as described in Example 9. Yield: 75 mg (35%).

¹H NMR (CD₃OD+CDCl₃): δ 7.9 (m, 1H), 7.7 (d, 2H), 7.56.8 (m, 8H), 6.6 (m, 1H), 6.1 (s, 1H), 3.7 (s, 3H), 3.5 (s, 2H), 3.1 (m, 1H), 2.3 (s, 3H), 1.0 (d, 6H).

### Example 31

### 3,5-Dibromo-4-{2-[(N-{4-methylbenzenesulfonyl}amino)(3-methylphenyl)-methyl]-5-isopropyl-4-methoxyphenoxy}phenylacetic Acid (E31)

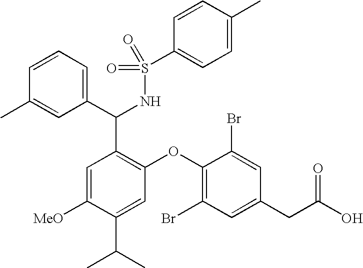

The compound was prepared according to the method described in Example 7 from 4-{2-[chloro(3-methylphenyl)methyl]-5-isopropyl-4-methoxyphenoxy}-3,5-dibromo-phenylacetic acid metyl ester, prepared as described in Example 7, Step 2, from thionyl chloride and 3,5-dibromo-4-{2-[hydroxy(3-methylphenyl)-methyl]-5-isopropyl-4-methoxyphenoxy}-phenylacetic acid, prepared as described in Example 9, and p-toluenesulfonamide. ¹H NMR (CDCl₃): δ 7.62 (d, 2H), 7.49 (d, 2H), 7.30-6.80 (m, 7H), 6.34 (s, 1H), 6.01 (s, 1H), 3.58 (s, 3H), 3.56 (s, 2H), 3.06 (m, 1H), 2.26 (s, 3H), 2.25 (s, 3H), 0.97 (dd, 6H).

### EXAMPLE 32

### 4-{2-[Benzylthio(3-methylphenyl)methyl]-5-isopropyl-4-methoxyphenoxy}-3,5-dibromophenylacetic Acid (E32)

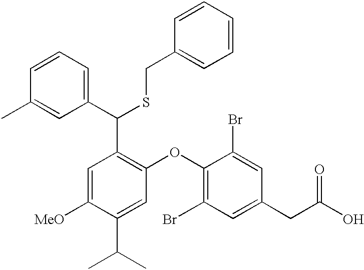

The title compound was prepared according to the procedure described in Example 20 from benzyl mercaptan. ¹H NMR (CDCl₃): δ 7.50 (m, 2H), 7.40-7.07 (m, 9H), 7.00 (m, 1H), 6.06 (s, 1H), 5.75 (s, 1H), 3.79 (s, 3H), 3.74 (s, 1H), 3.68 (s, 1H), 3.64 (s, 2H), 3.14 (m, 1H), 2.30 (s, 3H), 1.01 (dd, 6H).

### EXAMPLE 33

3,5-Dibromo-4-{2-[(2-furylmethylthio)(3-methylphenyl)methyl]-5-isopropyl-4-methoxyphenoxy}phenylacetic Acid (E33).

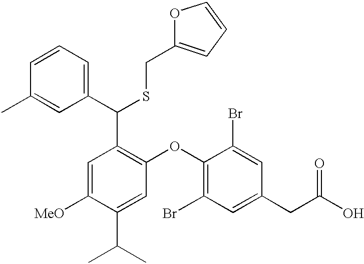

The compound was prepared as described in Example 20 from furfuryl mercaptan. ¹H NMR (CDCl₃): δ 7.48 (m, 2H), 7.45-6.95 (m, 6H), 6.23 (m, 1H), 6.16 (m, 1H), 6.05 (s, 1H), 5.82 (s, 1H), 3.80 (s, 3H), 3.75-3.66 (m, 2H), 3.64 (s, 2H), 3.13 (m, 1H), 2.29 (s, 3H), 1.00 (dd, 6H).

### EXAMPLE 34

### 4-{2-[Carboxymethylthio(3-methylphenyl)methyl]-5-isopropyl-4-methoxyphenoxy}-3,5-dibromo-phenylacetic Acid (E34)

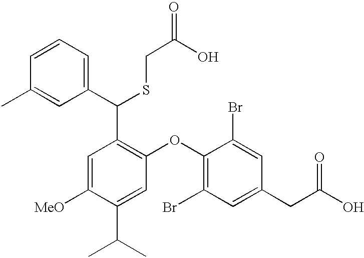

The compound was prepared as described in Example 20 and methyl mercaptoacetate.

¹H NMR (CDCl₃): δ 7.50 (m, 2H), 7.40-6.99 (m, 6H), 6.11 (s, 1H), 6.07 (s, 1H), 3.81 (s, 3H), 3.62 (s, 2H), 3.27 (d, 1H), 3.14 (d, 1H), 2.30 (s, 3H), 1.00 (dd, 6H).

### EXAMPLE 35

### 3,5-Dibromo-4-{2-[(3-methylphenyl)phenylthio)methyl]-5-isopropyl-4-methoxyphenoxy}phenylacetic Acid (E35)

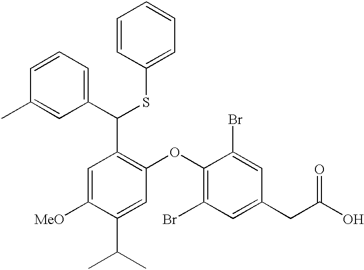

The compound was prepared according to the method described in Example 7, Step 3, from 4-{2-[chloro(3-methylphenyl)methyl]-5-isopropyl-4-methoxy-phenoxy}-3,5-dibromo-phenylacetic acid methyl ester, prepared as described in Example 7, Step 2, from thionyl chloride and 3,5-Dibromo-4-{2-[hydroxy(3-methylphenyl)-methyl]-5-isopropyl-4-methoxyphenoxy}-phenylacetic acid, prepared as described in Example 9, and thiophenol. ¹H NMR (CDCl₃): δ 7.60-6.90 (m, 12H), 6.33 (s, 1H), 6.11 (s, 1H), 3.77 (s, 3H), 3.64 (s, 2H), 3.15 (m, 1H), 2.33 (s, 3H), 1.00 (d, 6H).

### EXAMPLE 36

### 3,5-Dibromo-4-{2-[hydroxy(4-methylphenyl)methyl]-5-isopropyl-4-methoxyphenoxy}phenylacetic Acid (E36)

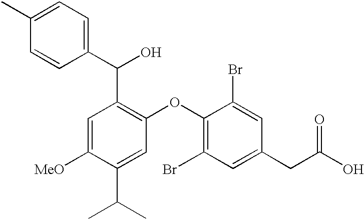

Step 1.

3,5-Dibromo-4-[5-isopropyl-4-methoxy-2-(4-methylbenzoyl)phenoxy]phenylacetic Acid Methyl Ester.

The compound was prepared as described in Example 1, Step 3, from 3,5-dibromo-4-(3-isopropyl-4-methoxyphenoxy)phenylacetic acid methyl ester (20.0 g, 42.3 mmol) and p-toluoyl chloride (22.9 g, 148.0 mmol). Yield: 16.2 g (65%).

Step 2.

3,5-Dibromo-4-{2-[hydroxy-(4-methylphenyl)methyl]5-isopropyl-4-methoxy-phenoxy}phenyl acetic Acid.

The compound was prepared as described in Example 1, Step 4, from 3,5-Dibromo-4-[5-isopropyl-2-(4-methylbenzoyl)-4-methoxyphenoxy]phenylacetic acid methyl ester (9.0 g, 15.3 mmol). Yield: 7.9 g (88%). ¹H NMR (CDCl₃): δ 7.53 (s, 2H), 7.41 (m, 2H), 7.15 (m, 2H), 6.91 (s, 1H), 6.33 (s, 1H), 6.11 (s, 1H), 3.74 (s, 3H), 3.64 (s, 2H), 3.15 (m, 1H), 2.32 (s, 3H), 1.01 (d, 6H).

### EXAMPLE 37

3,5-Dibromo-4-{2-[1-isopropoxy-1-(4-methylphenyl)methyl]-5-isopropyl-4-methoxyphenoxy}p henylacetic Acid (E37).

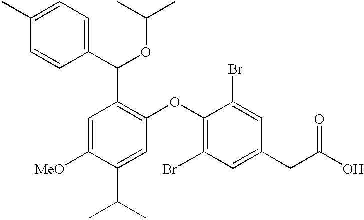

The compound was prepared as described in Example 11 from 3,5-dibromo-4-{2-[hydroxy-(4-methylphenyl)methyl]-5-isopropyl-4-methoxyphenoxy}phenylacetic acid, prepared as described in example 36. ¹H NMR (CDCl₃): δ 7.5 (s, 2H), 7.4 (d, 2H), 7,1 (d, 2H), 7,0 (s, 1H), 6,2 (s, 1H), 6.0 (s, 1H), 3.7 (m, 4H), 3.6 (s, 2H), 3.1 (m, 1H), 2.3 (s, 3H), 1.0 (d, 6H).

### EXAMPLE 38

### 3,5-Dibromo-4-{2-[(3-isopropylphenyl)hydroxymethyl]-5-isopropyl-4-methoxyphenoxy}phenylacetic Acid (E38)

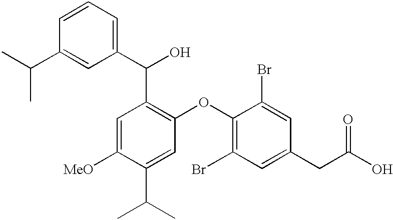

The compound was prepared as described in Example 1 from 3,5-dibromo-4-(3-isopropyl-4-methoxyphenoxy)phenylacetic acid methyl ester and 3-isopropylbenzoylchloride.

¹H NMR (CDCl₃): δ 7.59 (m, 2H), 7.35 (s, 1H), 7.30-7.00 (m, 4H), 6.34 (s, 1H), 6.10 (s, 1H), 3.79 (s, 3H), 3.67 (s, 2H), 3.15 (m, 1H), 2.85 (m, 1H), 1.18 (s, 6H), 0.98 (dd, 6H).

### EXAMPLE 39

### 3,5-Dibromo-4-{2-[(3-fluorophenyl)hydroxymethyl]-5-isopropyl-4-methoxyphenoxy}phenylacetic Acid (E39)

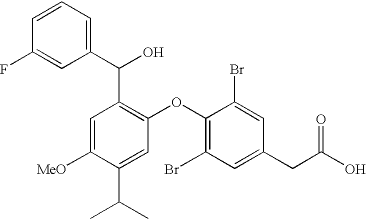

The compound was prepared as described in Example 1 from 3,5-dibromo-4-(3-isopropyl-4-methoxyphenoxy)phenylacetic acid methyl ester and 3-fluorobenzoylchloride.

¹H NMR (CDCl₃): δ 7.5 (s, 2H), 7.3-7.2 (m, 3H) 6.9 (m, 1H), 6.8 (s, 1H), 6.3 (s, 1H), 6.1 (s, 1H), 3.7 (s, 3H), 3.5 (s, 2H), 3.1 (m, 1H), 1.0 (d, 6H).

### EXAMPLE 40

### 3,5-Dibromo-4-{2-[hydroxy(3-iodophenyl)methyl]-5-isopropyl-4-methoxyphenoxy}phenylacetic Acid (E40)

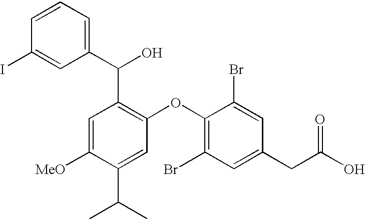

The compound was prepared as described in Example 1 from 3,5-dibromo-4-(3-isopropyl-4-methoxyphenoxy)phenylacetic acid methyl ester and 3-iodobenzoylchloride. ¹H NMR (CDCl₃): δ 7.9 (s, 1H), 7.6-7.5 (m, 4H), 7.1 (t, 1H), 6.9 (s, 1H), 6.3 (s, 1H), 6.1 (s, 1H), 3.8 (s, 3H), 3.6 (s, 2H), 3.2 (m, 1H), 1.0 (d, 6H).

### EXAMPLE 41

### 3,5-Dibromo-4-{2-[hydroxy(3-trifluorophenyl)methyl]-5-isopropyl-4-methoxyphenoxy}phenylacetic Acid (E41)

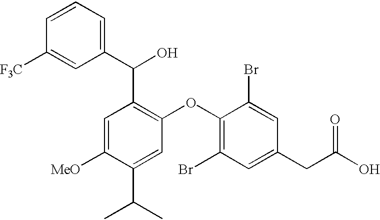

The compound was prepared as described in Example 1 from 3,5-dibromo-4-(3-isopropyl-4-methoxyphenoxy)-phenylacetic acid methyl ester and 3-trifluorobenzoylchloride.

¹H NMR (CDCl₃): δ 7.8 (s, 1H), 7.7 (d, 1H), 7.5-7.4 (m, 4H), 6.9 (s, 1H), 6.4 (s, 1H), 6.1 (s, 1H), 3.7 (s, 3H), 3.6 (s, 2H), 3.2 (m, 1H), 1.0 (d, 6H).

### EXAMPLE 42

### 3,5-Dibromo-4-{5-isopropyl-4-methoxy-2-[methoxy-(3-trifluorophenyl)-methyl]phenoxy}phenylacetic Acid (E42)

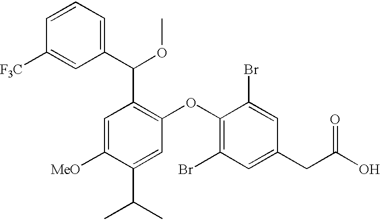

The compound was prepared as described in Example 10 from 3,5-dibromo-4-{2-[hydroxy(3-trifluorophenyl)methyl]-5-isopropyl-4-methoxyphenoxy}-phenyla cetic acid, prepared as described in Example 41. ¹H NMR (CDCl₃): δ 7.8 (s, 1H), 7.7 (d, 1H), 7.5 (s, 2H) 7.57.4 (m, 2H), 6.9 (s, 1H), 6.4 (s, 1H), 6.1 (s, 1H), 5.9 (s, 1H) 3.8 (s, 3H), 3.7 (s, 2H), 3.5 (s, 3H), 3.1 (m, 1H), 1.0 (d, 6H).

### EXAMPLE 43

3,5-Dibromo-4-{2-[hydroxy(3-trifluoromethoxyphenyl)methyl]-5-isopropyl-4-methoxyphenoxy}phenylacetic Acid (E43).

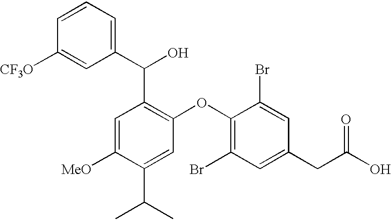

The compound was prepared as described in Example 1 from 3,5-dibromo-4-(3-isopropyl-4-methoxyphenoxy)phenylacetic acid methyl ester and 3-trifluoromethoxybenzoyl chloride. ¹H NMR (CDCl₃): δ 7.5 (s, 2H), 7.5-7.3 (m, 3H), 7.1 (d, 1H), 6.8 (s, 1H), 6.4 (s, 1H), 6.1 (s, 1H), 3.8 (s, 3H), 3.7 (s, 2H), 3.2 (m, 1H), 1.0 (d, 6H).

### EXAMPLE 44

3,5-Dibromo-4-{2-[(3-dimethylaminophenyl)hydroxymethyl]-5-isopropyl-4-methoxyphenoxy}phenylacetic Acid (E44).

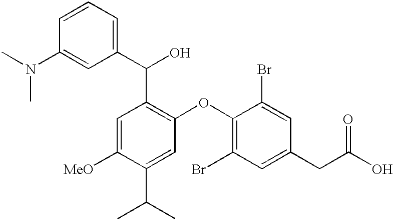

Step 1.

3,5-Dibromo-4-[2-(3-iodobenzoyl)-5-isopropyl-4-methoxyphenoxy]phenylacetic Acid.

The compound was prepared as described in Example 1 from 3,5-dibromo-4-(3-isopropyl-4-methoxyphenoxy)phenylacetic acid methyl ester and 3-iodobenzoylchloride.

¹H NMR (CDCl₃): δ 8.26 (m, 1H), 7.97(m, 1H), 7.85 (m, 1H), 7.19 (m, 1H), 7.02 (s, 1H), 6.24 (s, 1H), 3.83 (s, 3H), 3.60 (s, 2H), 3.24 (m, 1H), 1.06 (d, 6H)

Step 2.

3,5-Dibromo-4-[2- (3-dimethylaminobenzoyl)-5-isopropyl-4-methoxyphenoxy]phenylacetic Acid.

A mixture of 3,5-Dibromo-4-[2-(3-iodobenzoyl)-5-isopropyl-4-methoxyphenoxy]phenylacetic acid (1.0 g, 1.45 mmol), tris(dibenzylideneacetone)dipalladium (13.3 mg, 14 μmol), 2,2′-bis-(diphenylphosphino)-1,1′-binaphtyl (27.1 mg, 42 μmol), sodium tert-butoxide (195 mg, 2.0 mmol), 18-crown-6 (538 mg, 2.0 mmol) and 2M dimethylamine in THF (1.74 mL, 3.48 mmol) was stirred at room temperature for 20 h. Filtration, concentration and purification by chromatography gave the title compound.

Step 3.

3,5-Dibromo-4-{2-[(3-dimethylaminophenyl)hydroxymethyl]-5-isopropyl-4-methoxyphenoxy}phenylacetic Acid.

The compound was prepared as described in Example 1, Step 4. Yield: 0.7 mg (28%).

¹H NMR (CD₃OD): δ 7.8-6.7 (m, 7H), 6.3 (s, 1H), 6.1 (s, 1H), 3.8 (s, 3H), 3.6 (s, 2H), 2.9 (s, 6H), 2.4 (s, 3H), 1.0 (m, 6H).

### EXAMPLE 45

3,5-Dibromo-4-{2-[(3,5-dimethylphenyl)hydroxymethyl]-5-isopropyl-4-methoxy-phenoxy}phen ylacetic Acid (E45).

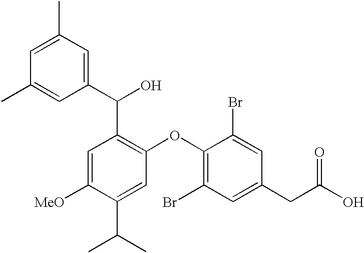

Step 1.

3,5-Dibromo-4-[2-(3,5-dimethylbenzoyl)-5-isopropyl-4-methoxyphenoxy]phenylacetic Acid Methyl Ester.

The compound was prepared as described in Example 1, Step 3, from 3,5-Dibromo-4-(3-isopropyl-4-methoxyphenoxy)phenylacetic acid methyl ester (570 mg, 1.19 mmol) and 3,5-dimethylbenzoyl chloride (602 mg, 3.58 mmol). Yield 550 mg (80%).

Step 2.

3,5-Dibromo-4-[2-(3,5-dimethylbenzoyl)-5 isopropyl-4-methoxyphenoxy]phenylacetic Acid.

The compound was prepared as described in Example 3, Step 2, from 3,5-dibromo-4-[2-(3,5-dimethylbenzoyl)-5-isopropyl-4-methoxyphenoxy]phenyl acetic acid methyl ester (150 mg, 0.25 mmol). Yield: 29 mg (20%). ¹H NMR (CDCl₃): δ 7.62 (s, 2H), 7.45 (s, 2H), 7.17 (s, 1H), 6.99 (s, 1H), 6.25 (s, 1H), 3.82 (s, 3H), 3.59 (s, 2H), 3.30-3.15 (m, 1H), 2.35 (d, 6H), 1.06 (s, 6H).

Step 3.

3,5-Dibromo-4-{2-[(3,5-dimethylphenyl)hydroxymethyl]-5-isopropyl-4-methoxy-phenoxy}phe nylacetic Acid.

The compound was prepared as described in Example 3, Step 3, from 3,5-dibromo-4-[2-(3,5-dimethylbenzoyl)-5-isopropyl-4-methoxyphenoxy]phenylacetic acid (383 mg, 0.65 mmol). Yield: 349 mg (91%). ¹H NMR (CDCl₃): δ 7.54 (s, 2H), 7.14 (s, 2H), 6.94 (s, 1H), 6.89 (s, 1H), 6.32 (s, 1H), 6.12 (s, 1H), 3.76 (s, 3H), 3.64 (s, 2H), 3.25-3.05 (m, 1H), 2.26 (s, 6H), 1.01 (d, 6H).

### EXAMPLE 46

### 4-{2-[Cyclohexyloxy(3,5-dimethylphenyl)methyl]-5-isopropyl-4-methoxyphenoxy}-3,5-dibromophenylacetic Acid (E46)

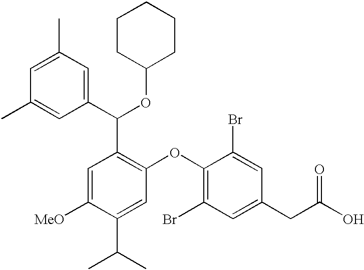

The compound was prepared as described in Example 12 from 3,5-dibromo-4-{2-[(3,5-dimethylphenyl)hydroxymethyl]-5-isopropyl-4-methoxyphenoxy}pheny l)-acetic acid (100 mg, 0.17 mmol), prepared as described in Example 45. Yield 28 mg (25%). ¹H NMR (CDCl₃): δ 7.55 (s, 2H), 7.18 (s, 2H), 7.02 (s, 1H), 6.82 (s, 1H), 6.19 (s, 1H), 6.06 (s, 1H), 3.75 (s, 3H), 3.65 (s, 2H), 3.50-3.35 (m, 1H), 3.20-3.05 (m, 1H), 2.27 (s, 6H), 2.20-1.90 (m, 2H), 1.90-1.60 (m, 2H), 1.60-1.35 (m, 3H), 1.35-1.10 (m, 3H), 1.02 (d, 6H).

### EXAMPLE 47

### 3,5-Dibromo-4-{2-[(3,5-difluorophenyl)hydroxymethyl]-5-isopropyl-4-methoxyphenoxy}phenylacetic Acid (E47)

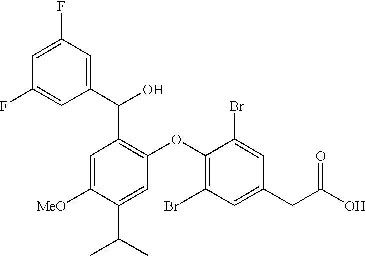

Step 1.

3,5-Dibromo-4-[2-(3,5-difluorobenzoyl)-5-isopropyl-4-methoxyphenoxy]phenylacetic Acid Methyl Ester.

The compound was obtained by the method described in Example 1, Step 3, from 3,5-Dibromo-4-(3-isopropyl-4-methoxyphenoxy)phenylacetic acid methyl ester (100 mg, 0.21 mmol) and 3,5-difluorobenzoylchloride (130 mg, 0.74 mmol). Yield: 40 mg (31%).

Step 2.

3,5-Dibromo-4-[2-(3,5-difluorobenzoyl)-5-isopropyl-4-methoxyphenoxy]phenylacetic Acid.

The compound was prepared as described in Example 3, Step 2, from 3,5-dibromo-4-[2-(3,5-difluorobenzoyl)-5-isopropyl-4-methoxyphenoxy]phenylacetic acid methyl ester (40 mg, 0.06 mmol). Yield: 36 mg (92%).

Step 3.

3,5-Dibromo-4-{2-[(3,5-difluorophenyl)hydroxymethyl]-5-isopropyl-4-methoxyphenoxy}phenylacetic Acid.

The compound was prepared as described in Example 3, Step 3 from 3,5-Dibromo-4-[2-(3,5-difluorobenzoyl)-5-isopropyl-4-methoxyphenoxy]phenylacetic acid (19 mg, 0.03 mmol). Yield: 18 mg (96%). ¹H NMR (CDCl₃): δ 7.53 (s, 2H), 7.07 (m, 2H), 6.87 (s, 1H), 6.67 (m, 1H), 6.26 (s, 1H), 6.12 (s, 1H), 3.77 (s, 3H), 3.64 (s, 2H), 3.16 (m, 1H), 1.02 (d, 6H).

### EXAMPLE 48

### 4-{[2-(3-chloro-2-fluorophenyl)hydroxymethyl]-5-isopropyl-4-methoxyphenoxy}-3,5-dibromophenylacetic Acid (E48)

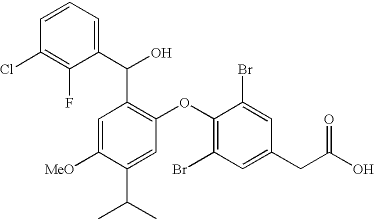

Step 1.

4-[2-(3-chloro-2-fluorobenzoyl)-5-isopropyl-4-methoxyphenoxy]-3,5-dibromophenylacetic Acid Methyl Ester.

The compound was prepared as described in Example 1, Step 3, from 3,5-dibromo-4-(3-isopropyl-4-methoxyphenoxy)phenylacetic acid methyl ester (400 mg, 0.84 mmol) and 3-chloro-2-fluorobenzoyl chloride (620 mg, 3.21 mmol). Yield: 510 mg, (96%).

Step 2.

4-[2-(3-chloro-2-fluorobenzoyl)-5-isopropyl-4-methoxyphenoxy]-3,5-dibromophenylacetic Acid.

The compound was prepared as described in Example 3, Step 2, from 3,5-Dibromo-4-[2-(3-chloro-2-fluorobenzoyl)-5-isopropyl-4-methoxyphenoxy]phenylacetic acid methyl ester (510 mg, 0.81 mmol). Yield: 354 mg (71%). ¹H NMR (CDCl₃): δ 7.95-7.85 (m, 1H), 7.70-7.05 (m, 5H), 6.17 (s, 1H), 3.86 (s, 3H), 3.59 (s, 2H), 3.30-3.15 (m, 1H), 1.03 (d, 6H).

Step 3.

4-{[2-(3-chloro-2-fluorophenyl)hydroxymethyl]-5-isopropyl-4-methoxyphenoxy}-3,5-dibromop henylacetic Acid.

The compound was prepared as described in Example 3, Step 3, from 3,5-dibromo-4-[2-(3-chloro-2-fluorobenzoyl)-5-isopropyl-4-methoxyphenoxy]phenylacetic acid (42 mg, 0.07 mmol. Yield: 37 mg (87%). ¹H NMR (CDCl₃): δ 7.95-7.85 (m, 1H), 7.70-7.05 (m, 4H), 6.91 (s, 1H), 6.53 (s, 1H), 6.10 (s, 1H), 3.76 (s, 3H), 3.65 (s, 2H), 3.30-3.10 (m, 1H), 0.98 (d, 6H).

### EXAMPLE 49

### 4-{[2-(3-Chloro-2-fluorophenyl)methoxymethyl-5-isopropyl-4-methoxyphenoxy}-3,5-dibromo-phenylacetic Acid (E49)

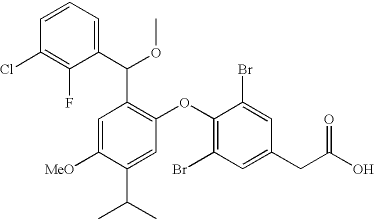

The compound was prepared as in Example 10 from 4-{2-[(3-chloro-2-fluorophenyl)-hydroxymethyl]-5-isopropyl-4-methoxy}phenoxy-3,5-dibromophenylacetic acid, prepared as described in Example 48. ¹H NMR (CDCl₃): δ 7.53 (s, 1H), 7.48 (s, 1H), 7.3 (m, 2H), 7.1 (s, 1H), 7.0 (app. t, 1H) 6.11 (s, 1H), 6.07 (s, 1H), 3.8(s, 3H), 3.6 (s, 2H), 3.5 (s, 3H), 3.17 (m, 1H), 1.02 (d, 6H).

### EXAMPLE 50

3,5-Dibromo-4-[2-(4-fluoro-3-methylphenyl)hydroxymethyl)-5-isorpropyl-4-methparnhenoxyl[p henylacetic Acid (E50).

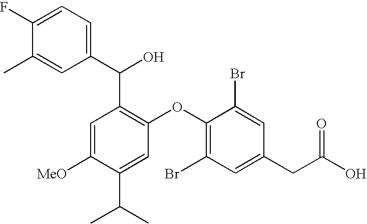

Step 1.

3,5-Dibromo-4-[2-(4-fluoro-3-methylbenzoyl)-5-isopropyl-4-methoxyphenoxy]phenylacetic Acid Methyl Ester.

The compound was prepared as described in Example 1, Step 3, from 3,5-dibromo-4-(3-isopropyl-4-methoxyphenoxy)phenylacetic acid methyl ester (400 mg, 0.84 mmol) and 4-fluoro-3-methylbenzoyl chloride (510 mg, 2.95 mmol). Yield: 440 mg (86%).

Step 2.

3,5-Dibromo-4-[2-(4-fluoro-3-methylbenzoyl)-5-isopropyl-4-methoxyphenoxy]phenylacetic Acid.

The compound was prepared as described in Example 3, Step 2, from 3,5-Dibromo-4-[2-(4-fluoro-3-methylbenzoyl)-5-isopropyl-4-methoxyphenoxy]phenylacetic acid methyl ester (440 mg, 0.72 mmol). Yield: 319 mg (74%). ¹H NMR (CDCl₃): δ 7.95-7.80 (m, 2H), 7.45 (s, 2H), 7.10-7.00 (m, 2H), 6.25 (s, 1H), 3.82 (s, 3H), 3.58 (s, 2H), 3.30-3.15 (m, 1H), 2.22 (s, 3H), 1.05 (d, 6H).

Step 3.

3,5-Dibromo-4-[2-(4-fluoro-3-methylphenyl)hydroxymethyl)-5-isopropyl-4-methoxyphenoxy]-phenylacetic Acid.

The compound was prepared as described in Example 3, Step 3, from 3,5-dibromo-4-[2-(4-fluoro-3-methylbenzoyl)-5-isopropyl-4-methoxyphenoxy]phenylacetic acid acid (42 mg, 0.07 mmol). Yield: 25 mg (58%). ¹H NMR (CDCl₃): δ 7.53 (s, 2H), 7.40-7.25 (m, 2H), 7.00-6.95 (m, 2H), 6.28 (s, 1H), 6.10 (s, 1H), 3.75 (s, 3H), 3.65 (s, 2H), 3.25-3.10 (m, 1H), 2.25 (s, 3H), 1.00 (d, 6H).

### EXAMPLE 51

4-{5-(2-Cyclopentylethyl)-2-[hydroxy(3-methylphenyl)methyl]-4-methoxy-phenoxy}-3,5-dibro mophenylacetic Acid (E51).

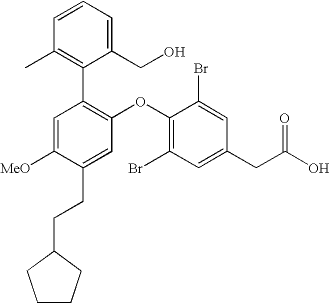

Step 1.

Bis(4-methoxyphenyl)iodonium Tetrafluoroborate.

Fuming nitric acid (45.0 ml, 962 mmol) was added dropwise to 62.0 mL of acetic acid anhydride cooled in a dry ice/CCl₄ bath. Iodine (21.0 g, 82.7 mmol) was added in one portion followed by dropwise addition of trifluoroacetic acid (76 mL, 986 mmol). The mixture was stirred at room temperature until the iodine was dissolved and purged with N₂ to remove nitrogen oxides. The mixture was concentrated, the residue dissolved in acetic anhydride (231 mL) and cooled in a dry ice/CCl₄ bath. A solution of anisole (45 g, 417 mmol) in acetic anhydride (277 mL) and trifluoroacetic acid (41 mL) was added dropwise with stirring. The mixture was left at room temperature 18 h and concentrated. The residue was taken up in MeOH (277 mL) and treated with 10% aqueous NaHSO₃ (277 mL) and 2M aqueous NaBF₄ (1.85 L). After the precipitate had aggregated, petroleum ether was added and the supernatant decanted. The precipitate was triturated with petroleum ether, filtered, washed with petroleum ether and dried at room temperature under vacuum to afford 16.4 g (46%) of the title compound.

Step 2.

3,5-Dibromo-4-(4-methoxyphenoxy)phenylacetic Acid Methyl Ester.

A solution of 3,5-dibromo-4-hydroxyphenylacetic acid methyl ester (5.00 g, 17.0 mmol) and triethylamine (1.87 g, 18.5 mmol) in CH₂Cl₂ (25 mL) was added dropwise to a mixture of bis(4-methoxyphenyl)iodonium tetrafluoroborate (9.91 g, 23.1 mmol) and copper bronze (1.27 g, 20.0 mmol) in CH₂Cl₂ (38 mL) at 0° C. The mixture was stirred in the dark for 7d and filtered through celite. The filtrate was concentrated and the residue purified by reversed phase chromatography to give 4.35 g (66%) of the title compound.

Step 3.

4-[3-(Cyclopentylacetyl)-4-hydroxyphenoxy]-3,5-dibromophenylacetic Acid Methyl Ester.

AlCl₃ (232 mg, 1.74 mmol) was added to a solution of 3,5-dibromo-4-(4-methoxyphenoxy)-phenylacetic acid methyl ester (150 mg, 0.35 mmol) and cyclopenthyl acetylchloride (179 mg, 1.22 mmol) in CH₂Cl₂ (1.5 mL) at 0° C. The mixture was stirred at room temperature for 4 h and poured into ice. After 10 min the layers were separated and the aqueous phase was extracted twice with EtOAc. The combined extracts were washed with brine and dried over MgSO₄. Concentration and purification by chromatography gave 114 mg (64%) of the title compound.

Step 4.

4-[3-(Cyclopentylacetyl)-4-methoxyphenoxyj-3,5-dibromophenylacetic Acid Methyl Ester.

K₂CO₃ (94 mg, 0.71 mmol) followed by Me₂SO₄ (68 mg, 0.54 mmol) was added to a solution of 4-[3-(cyclopentylacetyl)-4-hydroxyphenoxy]-3,5-dibromophenylacefic acid methyl ester (110 mg, 0.42 mmol) in acetone (3 mL). The mixture was stirred at room temperature for 16 h and concentrated. The residue was dissolved in water and the solution was extracted twice with EtOAc. The combined extracts were dried with MgSO₄, concentrated and purified by chromatography to give 165 mg (71%) of the title compound.

Step 5.

4-[3-(2-Cyclopentylethyl)-4-methoxyphenoxy]-3,5-dibromophenylacetic Acid Methyl Ester.

NaBH₄ (126 mg, 3.33 mmol) was added in one portion to a solution of 4-[3-(cyclopentylacetyl)-4-methoxyphenoxy]-3,5-dibromophenylacetic acid methyl ester (90 mg, 0,17 mmol) in a ⅔ mixture of TFA/CH₂Cl₂ (3 mL) at 0° C. The mixture was stirred at 0° C. for 15 min and allowed to reach room temperature. After 2 h at room temperature, the mixture was neutralized with NaHCO₃ (aq., sat). The layers were separated and the aqueous phase was extracted twice with EtOAc. The combined extracts were dried over MgSO₄, concentrated and purified by chromatography to give 45 mg (51%) of the title compound.

Step 6.

4-[5(2-Cyclopentylethyl)-4-methoxy-2-(3-methylbenzoyl)phenoxy]-3,5-dibromophenylacetic Acid Methyl Ester.

The compound was prepared as described in Example 1, Step 3, from 4-[3-(2-cyclopentylethyl)-4-methoxyphenoxy]-3,5-dibromophenylacetic acid methyl ester (45 mg, 0.09 mmol) and m-toluoyl chloride (81 mg, 0.43 mmol). Yield: 26 mg (48%).

Step 7.

4-[5-(2-Cyclopentylethyl)-4-methoxy-2-(3-methylbenzoyl)phenoxy]-3,5-dibromophenylacetic Acid.

The compound was prepared as described in Example 3, Step 2, from 4-[5-(2-cyclopentylethyl)-4-methoxy-2-(3-methylbenzoyl)phenoxy]-3,5-dibromophenylacetic acid methyl ester (25 mg, 0.04 mmol). Yield: 22 mg (92%). ¹H NMR (CDCl₃): δ 7.82 (m, 2H), 7.45 (s, 2H), 7.31 (m, 2H), 7.00 (s, 1H), 6.18 (s, 1H), 3.81 (s, 3H), 3.59 (s, 2H), 2.50 (t, 2H), 2.39 (s, 3H), 1.68-1.05 (m, 11H).

Step. 8.

4-{5-(2-Cyclopentylethyl)-2-[hydroxy(3-methylphenyl)methyl]-4-methoxyphenoxy}-3,5-dibro mophenylacetic Acid

The compound was prepared as described in Example 3, Step 3, from 4-[5-(2-cyclopentylethyl)-4-methoxy-2-(3-methylbenzoyl)phenoxy]-3,5-dibromophenylacetic acid (10 mg, 16 μmol). Yield: 8 mg (80%). ¹H NMR (CDCl₃): δ 7.51 (s, 2H), 7.35-7.18 (m, 3H), 7.05 (m, 1H), 6.89 (s, 1H), 6.31 (s, 1H), 6.03 (s, 1H), 3.72 (s, 3H), 3.61 (s, 2H), 2.43 (m, 2H), 2.32 (s, 3H), 1.64-1.02 (m, 11H).

### EXAMPLE 52

### 4-{2-[hydroxy(3-methylphenyl)methyl]-5-iodo-4-methoxyphenoxy}-3,5-dibromophenylacetic Acid (E52)

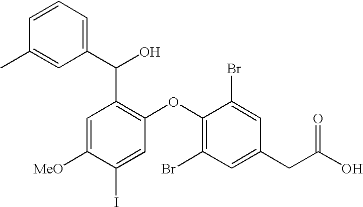

Step 1.

4-[3-iodo-4-methoxyphenoxy]-3,5-dibromophenylacetic Acid Methyl Ester.

Iodine (64.9 mg, 0.26 mmol) and AgSO₄ (79.8 mg, 0.26 mmol) was added to a solution of 3,5-dibromo-4-7.8-7.4 (m, 5H), 7.3-7.2 (m, 1H), 7.1-7.0 (m, 1H), 6.0 (s, 1H), 5.6 (s, 1H), 3.7 (s, 3H), 3.6 (s, 2H), 3.2-2.9 (m, 5H), 2.3 (s, 3H), 1.9-1.7 (m, 4H), 1.6-1.4 (m,2H), 1.0-0.7 (m, 6H) (4-methoxyphenoxy)phenylacetic acid methyl ester (100 mg, 0.23 mmol), prepared as described in Example 51, Step 2, in methanol at −78° C. The cooling bath was removed and the mixture was stirred for 4 h, filtered through silica gel and concentrated. Purification by chromatography gave 122 mg (94%) of the title compound. ¹H NMR (CDCl₃): δ 7.51 (s, 2H), 7.26 (d, 1H), 6.72 (d, 1H), 6.71 (s, 1H), 3.81 (s, 3H), 3.74 (s, 3H), 3.60 (s, 2H).

Step 2.

4-[5-iodo-4-methoxy-2-(3-methylbenzoyl)phenoxy]-3,5-dibromophenylacetic Acid Methyl Ester

Silver triflate (88 mg, 0.34 mmol) was added to a solution of m-toluoyl chloride (45 μl, 0.34 mmol) in dry 1,2-dichloroethane (0.5 ml) at 0° C. under protection from light. The cooling bath was removed and the mixture was stirred for 1 h. The mixture was then filtered, using a canula, to a flask wrapped in aluminium foil containing 43-iodo-4-methoxyphenoxy]-3,5-dibromophenylacetic acid methyl ester (38.2 mg, 0.069 mmol). After 5 d at room temperature, NaHCO₃ (sat.) was added, the aqueous phase was separated and extracted four times with CH₂Cl₂. The combined extracts were dried over Na₂SO₄, concentrated. Purification by chromatography gave 11.8 mg (25%) of the title compound. ¹H NMR (CDCl₃): δ 7.70-7.87 (m, 2H), 7.45 (s, 2H), 7.30-7.43 (m, 2H), 6.95 (s, 1H), 6.81 (s, 1H), 3.87 (s, 3H), 3.70 (s, 3H), 3.55 (s, 2H), 2.39 (s, 3H).

Step 3.

4-[5-iodo-4-methoxy-2-(3-methylbenzoyl)phenoxy]-3,5-dibromophenylacetic Acid.

The compound was prepared as described in Example 3, Step 2, from 4-[5-iodo-4-methoxy-2-(3-methylbenzoyl)phenoxyl-3,5-dibromophenylacetic acid methyl ester (11.8 mg, 0.018 mmol). Yield: 11.1 mg (96%). ¹H NMR (CDCl₃): δ 7.70-7.88 (m, 2H), 7.46 (s, 2H), 7.29-7.42 (m, 2H), 6.95 (s, 1H); 6.81 (s, 1H), 3.87 (s, 3H), 3.59 (s, 2H), 2.39 (s, 3H).

Step 4.

4-{2-[hydroxy(3-methylphenyl)methyl]-5-iodo-4-methoxyphenoxy}-3,5 dibromophenylacetic Acid.

The compound was prepared as described in Example 3, Step 3, from 4-[5-iodo-4-methoxy-2-(3-methylbenzoyl)phenoxy]-3,5-dibromophenylacetic acid (11.1 mg, 0.017 mmol). Yield: 10.6 mg (95%). ¹H NMR (CDCl₃): δ 7.52 (2s, 2H), 7.18-7.37 (m, 3H), 7.00-7.18 (m, 2H), 6.60 (s, 1H), 2.32 (s, 3H), 6.30 (s, 1H), 3.83 (s, 3H), 3.64 (s, 2H).

### EXAMPLE 53

4-{5-acetamido-2-[hydroxy(3-methylphenyl)methyl]4-methoxyphenoxy}-3,5-dibromophenylacetic Acid (E53).

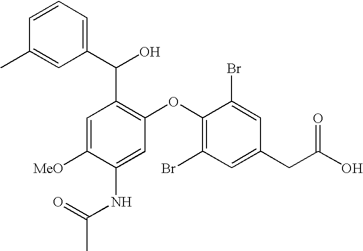

Step 1.

4-[4-methoxy-3-nitrophenoxy]-3,5-dibromophenylacetic Acid Methyl Ester.

Ice-cold nitric acid (65% v/v, 0.080 ml, 1.2 mmol) was added to a solution of 3,5-Dibromo-4-(4-methoxyphenoxy)phenylacetic acid methyl ester (50.3 mg, 0.117 mmol), prepared as described in Example 51, Step 2, in benzene at 0° C. The mixture was stirred at room temperature for 5 h and cooled to 0° C. NaHCO₃ (aq. sat.) was added, the layers separated and the aqueous phase extracted three times with CH₂Cl₂. The combined extracts were washed with brine and dried over Na₂SO4. Concentration and purification by chromatography gave 55.6 mg (100%) of the title compound. ¹H NMR (CDCl₃): δ 7.54 (s, 2H), 7.29 (d, 1H), 6.96-7.10 (m, 2H), 3.91 (s, 3H), 3.74 (s, 3H), 3.60 (s, 2H).

Step 2.

4-[3-amino-4-methoxyphenoxy]-3,5-dibromophenylacetic Acid Methyl Ester.

Sodiumdithionate (85%, 44.4 mg, 0.22 mmol) was added to a solution of 4-[4-methoxy-3-nitrophenoxy]-3,5-dibromophenylacetic acid methyl ester (20.6 mg, 0.043 mmol) in methanol (95%) and the mixture was heated to 60° C. for 22 h. Concentration and purification by chromatography gave 9.7 mg (50%) of the title compound. ¹H NMR (CDCl₃): δ 7.50 (s, 2H), 6.64(d, 1H), 6.30 (d, 1H), 6.08(dd, 1H), 4.15 (bs, 1H), 3.80 (s, 3H), 3.72 (s, 3H), 3.59 (s, 2H).

Step 3.

4-[3-acetamido-4-methoxyphenoxy]-3,5-dibromophenylacetic Acid Methyl Ester.

Acetyl chloride (0.012 ml, 0.17 mmol) was added to an ice-cold mixture of 4-[3-amino-4-methoxyphenoxy]-3,5-dibromophenylacetic acid methyl ester (25 mg, 0.056 mmol), triethylamine (0.039 ml, 0.28 mmol) and N,N-dimethyl-4-amino pyridine (cat.) in CH₂Cl₂ at 0° C. The cooling bath was removed and the mixture was stirred for 25 min followed by addition of water. Concentration and purification by chromatography gave 27.4 mg (100%) of the title compound. ¹H NMR (CDCl₃): δ 8.10 (d, 1H), 7.78 (bs, 1H), 7.50 (s, 2H), 6.72 (d, 1H), 6.39 (dd, 1H), 3.84 (s, 3H), 3.73 (s, 3H), 3.59 (s, 2H), 2.17 (s, 3H).

Step 4.

4-[5-acetamido-2-(3-methylbenzoyl)-4-methoxy-)phenoxy]-3,5-dibromophenylacetic Acid Methyl Ester.

The compound was prepared as described in Example 52 from 4-[3-acetamido-4-methoxyphenoxy]-3,5-dibromophenylacetic acid methyl ester (15.3 mg, 0.031 mmol) and m-toluoyl chloride (0.42 ml, 0.32 mmol) and silver triflate (80.6 mg, 0.31 mmol). Yield: 8 mg (42%). ¹H NMR (CDCl₃): δ 7.75-7.92 (m, 3H), 7.71 (s, 1H), 7.44 (s, 2H), 7.27-7.40 (m, 2H), 7.11 (s, 1H), 3.90 (s, 3H), 3.69 (s, 3H), 3.52 (s, 2H), 2.40 (s, 3H), 2.12 (s, 3H).

During the chromatographic purification, 4-[5-(3-methylbenzamido)-2-(3-methylbenzoyl)-4-methoxyphenoxy]-3,5-dibromophenylacetic acid methyl ester was also isolated; Yield: 3 mg (14%). ¹H NMR (CDCl₃): δ 8.60 (s, 1H), 7.74-7.90 (m, 3H), 7.52-7.71 (m, 2H), 7.45 (s, 2H), 7.28-7.41 (m, 4H), 7.17 (s, 1H), 3.96 (s, 3H); 3.69 (s 3H), 3.53 (s, 2H), 2.40 (s, 6H).

Step 5.

4-[5-acetamido-2-(3-methylbenzoyl)-4-methoxy-)phenoxy]-3,5-dibromophenylacetic Acid.

The compound was prepared as described in Example 3, Step 2, from 4-[5-acetamido-2-(3-methylbenzoyl)-4-methoxy-)phenoxy]-3,5-dibromophenylacetic acid methyl ester (8.0 mg, 0.013 mmol). Yield: 7.8 mg (100%). ¹H NMR (CDCl₃): δ 7.72-7.92 (m, 3H), 7.69 (s, 1H), 7.44 (s, 2H), 7.27-7.40 (m, 2H), 7.11 (s, 1H), 3.90 (s, 3H), 3.53 (s, 2H), 2.38 (s, 3H), 2.11 (s, 3H).

Step 6.

4-{5-acetamido-2-[hydroxy(3-methylphenyl)methyl]-4-methoxyphenoxy}-3,5-dibromophenylacetic Acid

The compound was prepared as described in Example 3, Step 3, from 4-15-acetamido-2-(3-methylbenzoyl)-4-methoxy-)phenoxy]-3,5-dibromophenylacetic acid (7.8 mg, 0.013 mmol). Yield: 4.9 mg (63%). ¹H NMR (CDCl₃): δ 7.72 (bs, 1H), 7.53 (m, 3H), 7.18-7.34 (m, 3H), 7.06 (d, 1H), 6.99 (s, 1H), 6.35 (s, 1H), 3.81 (s, 3H), 3.60 (s, 2H), 2.33 (s, 3H), 2.08 (s, 3H).

### EXAMPLE 54

### 4-{2-[Hydroxy(3-methylphenyl)methyl]-4-methoxy-5-(3-methylbenzamido)-phenoxy}-3,5-dibromophenylacetic Acid (E54)

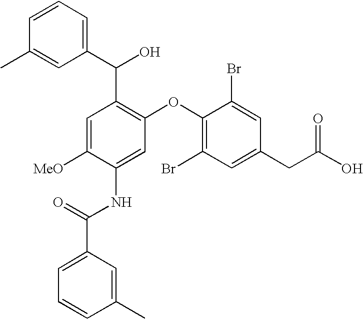

The compound was prepared as described in Example 53, Step 5 and 6, from 4-[5-(3-methylbenzamido)-2-(3-methylbenzoyl)-4-methoxyphenoxy]-3,5-dibromophenylacetic acid methyl ester, described in Example 53, Step 4. Yield: 2.5 mg (85%). ¹H NMR (CDCl₃): δ 8.45 (s, 1H), 7.67 (s, 1H), 7.46-7.50 (m, 4H), 7.18-7.40 (m, 5H), 7.02-7.12 (m, 2H), 6.39 (s, 1H), 3.88 (s, 3H), 3.61 (s, 2H), 2.37 (s, 3H), 2.32 (s, 3H).

### EXAMPLE 55

### 3,5-Dibromo-4-{4-hydroxy-2-[hydroxy(3-methylphenyl)methyl]-5-isopropylphenoxy}phenylacetic Acid (E65)

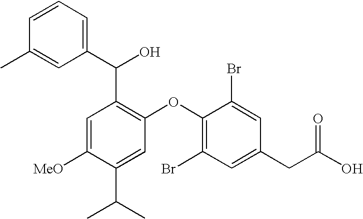

Step 1.

3,5-Dibromo-4-[4-hydroxy-5-isopropyl-2-(3-methylbenzoyl)phenoxy]phenylacetic Acid.

A 1M solution of BBr₃ in CH₂Cl₂ (12 mL, 12 mmol) was added slowly with a syringe to a stirred solution of 3,5-Dibromo-4-[5-isopropyl-4-methoxy-2-(3-methylbenzoyl)phenoxy]-phenylacetic acid (2 g, 3.5 mmol), prepared as described in Example 10, Step 2, in 20 mL CH₂Cl₂ at −20° C. After 15 min at −20° C. the solution was allowed to reach room temperature. After an additional 1 h at room temperature, the solution was poured onto ice and extracted three times with EtOAc. The combined extracts were dried over MgSO₄ and concentrated to give the title compound. ¹H NMR (CDCl₃): δ 7.8-7.7 (m, 2H), 7.5 (s, 2H), 7.4-7.3 (m, 2H), 6.9 (s, 1H), 6.2 (s, 1H), 3.6 (s, 2H), 3.3-3.2 (m, 1H), 2.4 (s, 3H), 1.1 (d, 6H).

Step 2.

3,5-Dibromo-4-{4-hydroxy-2-[hydroxy(3-methylphenyl)methyl]-5-isopropylphenoxy}-phenylacetic Acid.

The compound was prepared as described in Example 3, Step 3, from 3,5-dibromo-4-[4-hydroxy-5-isopropyl-2-(3-methylbenzoyl)phenoxy]phenylacetic acid.

¹H NMR (CDCl₃): δ 7.6 (s, 2H), 7.4-7.1 (m, 4H), 6.7 (s, 1H), 6.3 (s, 1H), 6.1 (s, 1H), 3.6 (s, 2H), 3.1 (m, 1H), 2.3 (s, 3H), 1.1 (d, 6H).

### EXAMPLE 56

### 3,5-Dibromo-4-{2-[hydroxy(3-methylphenyl)methyl]-4-isobutyloxy-5-isopropylphenoxy}phenylacetic Acid (E66)

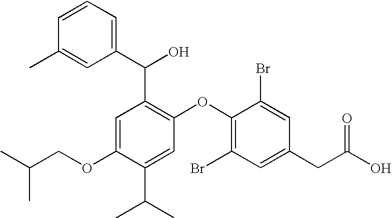

Step 1.

3,5-Dibromo-4-[4-hydroxy-5-isopropyl-2-(3-methylbenzoyl)phenoxy]phenylacetic Acid Methyl Ester.

Five drops of SOCl₂ was added to a solution of 3,5-dibromo-4-[4-hydroxy-5-isopropyl-2-(3-methylbenzoyl)phenoxy]phenylacetic acid (0.3 g, 0.52 mmol), prepared as described in Example 59, in 20 mL MeOH. The mixture was stirred at room temperature for 12 h and concentrated leaving the title compound Yield: 0.18 g, (60%). ¹H NMR (CDCl₃): δ 7.9-7.7 (m, 2H), 7.5-7.1 (m, 4H), 6.9 (s, 1H), 6.2 (s, 1H), 4.7 (s, 3H), 4.5 (s, 2H), 3.1 (m, 1H), 2.4 (s, 3H), 1.1 (d, 6H)

Step 2.

3,5-Dibromo-4-[4-isobutyloxy-5-isopropyl-2-(3-methylbenzoyl)phenoxy]phenylacetic Acid.

A solution of diethyl azodicarboxylate (42 mg, 0.24 mmol) in anhydrous CH₂Cl₂ (1 mL) was added over 15 min to an ice-cooled, stirred mixture of 3,5-Dibromo-4-[4-hydroxy-5-isopropyl-2-(3-methylbenzoyl)phenoxy]phenylacetic acid methyl ester (70 mg, 0.12 mmol), PPh₃ (63 mg, 0.24 mmol) and isobutanol (22 μL, 0.24 mmol) in anhydrous CH₂Cl₂ (2 mL). The mixture was stirred at 0° C. for 12 h and 3d at room temperature and concentrated. The residue was dissolved in 30% NaOH/MeOH (2 mL) and heated at 40° C. for 12 h. The solution was allowed to cool, acidified with 1M HCl, concentrated and purified by HPLC to give the title compound. ¹H NMR (CDCl₃): δ 7.9-7.7 (m, 2H), 7.5 (s, 2H), 7.4-7.3 (m, 2H), 7.0 (s, 1H), 6.2 (s, 1H), 3.8 (d, 2H), 3.6 (s, 2H), 3.3-3.2 (m, 1H), 2.4 (s, 3H), 2.2-2.0 (m, 1H), 1.1 (d, 6H), 1.0 (d, 6H).

Step 3.

3,5-Dibromo-4-{2-[hydroxy(3-methylphenyl)methyl]-4-isobutyloxy-5-isopropylphenoxy}-phenylacetic Acid.

The compound was prepared as described in Example 3, Step 3, from 3,5-Dibromo-4-[4-isobutyloxy-5-isopropyl-2-(3-methylbenzoyl)phenoxy]phenylacetic acid.

¹H NMR (CDCl₃): δ 7.5 (s, 2H), 7.47.0 (m, 4H), 6.9 (s, 1H), 6.3 (s, 1H), 6.1 (s, 1H), 3.8-3.6 (m, 4H), 3.2 (m, 1H), 2.3 (s, 3H), 2.1-2.0 (m, 1H), 1.1-0.9 (m, 12H).

### EXAMPLE 57

3,5-Dibromo-4-{4-[2-fluoroethoxyl-2-[hydroxy-(3-methylphenyl)methyl]-5-isopropyl-phenoxy}phenylacetic Acid (E57).

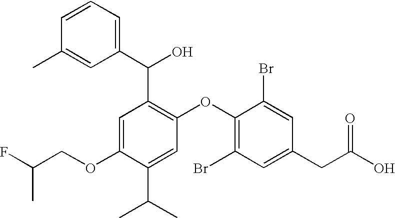

The compound was prepared according to the procedure described in Example 56 from 2-fluoroethanol. ¹H NMR (CDCl₃): δ 7.76.9 (m, 7H), 6.3 (s, 1H), 6.1 (s, 1H), 4.8 (m, 1H), 4.6 (m, 1H), 4.2 (m, 1H), 4.1 (m, 1H), 3.7 (s, 2H), 3.2 (m, 1H), 2.4 (s, 3H), 1.1-0.9 (m, 6H).

### EXAMPLE 58

### 3,5-Dibromo-4-{2-[hydroxy(3-methylphenyl)methyl]-5-isopropyl-4-methoxyphenoxy}phenylpropionic Acid (E58)

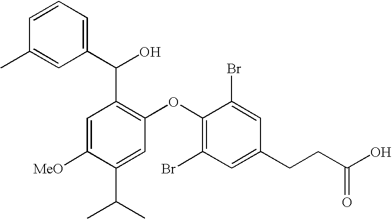

The compound was prepared according to the method described in Example 1 from 3,5-dibromo-4-hydroxyphenylpropionic acid methyl ester. ¹H NMR (CDCl₃): δ 7.44 (s, 2H), 7.29-7.37 (m, 2H), 6.98-7.22 (m, 2H), 6.88 (s, 1H), 6.32 (s, 1H), 6.10 (s, 1H), 3.73 (s, 3H), 3.15 (m, 1H); 2.93 (t, 2H), 2.66 (t, 2H), 2.33 (s, 3H), 1.10 (d, 6H).

The compounds of the present invention according to the general formula I exhibits an affinity for the glucocorticoid receptor in the range between 0.1 and 5000 nM.

1. A compound according to the formula I: 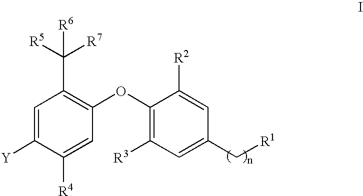
wherein:
R¹ is selected from:
COOH, C(O)NHOH, C(O)COOH, SO₃H, P(O)(OH)(OR⁸), P(O)(OH)[N(R⁹)(R¹⁰)], and heteroaryl, wherein any heteroaryl residue may be optionally substituted in one or more positions independently of each other by a group selected from C₁₋₆-alkyl, perfluoro-C₁₋₆-alkyl, halogen, cyano, nitro, R⁸O, R⁸S, R⁸S(O), R⁸S(O)₂ and (R⁹)(R¹⁰)N;
R² and R³ are independently of each other selected from:
hydrogen, halogen, C₁₋₆-alkyl, hydroxy, C₁₋₆-alkoxy, C₁₋₆-alkylthio, halo-C₁₋₆-alkyl, perfluoro-C₁₋₆-alkyl, halo-C₁₋₆-alkyloxy, perfluoro-C₁₋₆-alkyloxy, and halo-C₁₋₆-alkylthio, provided that one of R² or R³ is other than hydrogen;
R⁴, R⁵, R⁶ and R⁷ are independently of each other selected from:
(i) C₁₋₁₂-alkyl and perfluoro-C₁₋₆-alkyl, wherein any residues herein may be optionally substituted in one or more positions independently of each other by a group selected from A;
(ii) C₃₋₈-cycloalkyl, C₂₋₆-alkenyl, and C₂₋₆-alkynyl, wherein any residues herein may be optionally substituted in one or more positions independently of each other by a group selected from B;
R⁴ and R⁵ are optionally, and independently of each other, selected from:
(iii) C₃₋₈-heterocycloalkyl, optionally substituted by a group selected from B;
(iv) aryl and heteroaryl, wherein any residues herein may be optionally substituted in one or more positions independently of each other by a group selected from C;
R⁴ is optionally selected from:
halogen, R⁸O, R⁸S, R⁸S(O), R⁸S(O)₂, (R⁹)(R¹⁰)N, R⁸C(Z)N(R¹¹), (R⁹)(R¹⁰)NC(Z)N(R¹¹), R⁸S(O)₂N(R¹¹), and (R⁹)(R¹⁰)NS(O)₂N(R¹¹);
R⁶ and R⁷ are optionally, and independently of each other, selected from:
hydrogen, halogen, R⁸O, R⁸S, R⁸S(O), R⁸S(O)₂, (R⁹)(R¹⁰)N, R⁸C(Z)O, R⁸OC(Z)O, R⁸C(Z)N(R¹¹), R⁸OC(Z)N(R¹¹), R⁸S(O)ₙO, (R⁹)(R¹⁰)NC(Z)O, (R⁹)(R¹⁰)NS(O)₂O, R⁸S(O)₂N(R¹¹), and (R⁹)(R¹⁰)NS(O)₂N(R¹¹), provided that R⁸ is not hydrogen in R⁸OC(Z)O, R⁸S(O)ₙO, and R⁸S(O)₂N(R¹¹), and that only one of R⁶ and R⁷ is hydrogen, and that if R⁶ is HO, R⁷ is hydrogen, and that if R7 is HO, Re is hydrogen;
R⁸, R⁹, R¹⁰ and R¹¹ are independently of each other selected from:
(v) hydrogen,
(vi) C₁₋₁₂-alkyl and perfluoro-C₁₋₆-alkyl, wherein any residues herein may be optionally substituted in one or more positions independently of each other by a group selected from A;
(vii) C₃₋₈-cycloalkyl, C₂₋₆-alkenyl, C₂₋₆-alkynyl, and C₃₋₄-heterocycloalkyl, wherein any residues herein may be optionally substituted in one or more positions independently of each other by a group selected from B;
(viii) aryl and heteroaryl, wherein any residues herein may be optionally substituted in one or more positions independently of each other by a group selected from C;
or where any pair of R⁸, R⁹, R¹⁰ and R¹¹ together with the atom or atoms to which they are bound form a ring having 3-7 ring members, and which ring optionally contain 1-3 heteroatoms, or 1-3 double bonds, and which optionally is substituted by a group selected from B;
A is selected from:
halogen, perfluoro-C₁₋₆-alkyl, C₃₋₈-cycloalkyl, C₂₋₆-alkenyl, C₂₋₆-alkynyl, aryl, C₃₋₈-heterocycloalkyl, heteroaryl, cyano, nitro, azido, Z, R⁸O, R⁸C(Z), R⁸C(Z)O, R⁸OC(Z), R⁸S, R⁸S(O), R⁸S(O)₂, R⁸S(O)₂O, R⁸OS(O)₂, (R⁹)(R¹⁰)N, (R⁹)(R¹⁰)NC(Z), (R⁹)(R¹⁰)NC(Z)O, R⁸C(Z)N(R¹¹), R⁸OC(Z)N(R¹¹), (R⁹)(R¹⁰)NC(Z)N(R¹¹), (R⁹)(R¹⁰)NS(O)₂, R⁸S(O)₂N(R¹¹), (R⁹)(R¹⁰)NS(O)₂N(R¹¹), and R⁸SC(Z)N(R¹¹), wherein any perfluoro-C₁₋₆alkyl, C₃₋₈-cycloalkyl, C₂₋₆-alkenyl, C₂₋₆-alkynyl, and C₃₋₈-heterocycloalkyl residue is optionally substituted in one or more positions independently of each other by a group selected from B, and also wherein any aryl and heteroaryl residue is optionally substituted in one or more positions independently of each other by a group selected from C;
B is defined as:
A, or a C₁₋₆-alkyl optionally substituted in one or more positions independently of each other by a group selected from D, provided that if B is directly attached to a double or to a triple bond, or to a carbon directly attached to a heteroatom, B is not HO, HS, R⁹HN, (R⁹)(R¹⁰)NC(Z)NH, (R⁹)(R¹⁰)NS(O)₂NH, or R⁸S(O)₂NH, and also provided that if B is directly attached to a double or to a triple bond, B is not Z;
C is defined as:
A, or a C₁₋₆-alkyl optionally substituted in one or more positions independently of each other by a group selected from D, provided that C is not Z;
D is selected from:
halogen, cyano, nitro, azido, Z, R⁸O, R⁸C(Z), R⁸C(Z)O, R⁸OC(Z), R⁸S, R⁸S(O), R⁸S(O)₂, R⁸S(O)₂O, R⁸OS(O)₂, (R⁹)(R¹⁰)N, (R⁹)(R¹⁰)NC(Z), (R⁹)(R¹⁰)NC(Z)N(R¹¹), (R⁹)(R¹⁰)NS(O)₂, R⁸S(O)₂N(R¹¹), and (R⁹)(R¹⁰)NS(O)₂N(R¹¹);
Y is selected from:
hydrogen, halogen, hydroxy, C₁₋₆-alkoxy, halo-C₁₋₆-alkyloxy, perfluoro-C₁₋₆-alkyloxy, C₁₋₆-acyloxy, C₁₋₆-alkylthio, halo-C₁₋₆-alkylthio, perfluoro-C₁₋₆-alkylthio, Citalkylsulphonyloxy, azido, and (R⁹)(R¹⁰)N;
Z is a substituent connected by a double bond, and is selected from:
O═, S═, R⁸N═, (R⁹)(R¹⁰)NN═, R⁸ON═, (R⁹)(R¹⁰)NS(O)₂N═, NCN═, O₂NCH═, and (R⁹)(R¹⁰)C═;
n is 0, 1, 2 or 3;
or pharmaceutically acceptable salts, stereoisomers or prodrugs thereof.

2. A compound according to claim 1 wherein R¹ is COOH or heteroaryl.

3. A compound according to claim 2 wherein R¹ is COOH.

4. A compound according to any one of claims 1 to 3 wherein R² and R³ are independently of each other, halogen or C₁₋₆-alkyl.

5. A compound according to claim 4 wherein both R² and R³ are halogen.

6. A compound according to any one of claims 1 to 3 wherein both R² and R³ is bromine.

7. A compound according to any one of claims 1 to 6 wherein R⁴ is C₁₋₁₂-alkyl, C₃₋₈-cycloalkyl, C₂₋₆-alkenyl, C₂₋₆-alkynyl, C₃₋₈-heterocycloalkyl, halogen, (R⁹)(R¹⁰)N, or R⁸C(Z)N(R¹¹).

8. A compound according to claim 7 wherein R⁴ C₁₋₁₂-alkyl, halogen, (R⁹)(R¹⁰)N, or R⁸C(Z)N(R¹¹).

9. A compound according to claim 8 wherein R⁴ is C₁₋₁₂-alkyl.

10. A compound according to claim 9 wherein R⁴ is isopropyl.

11. A compound according to any one of claims 1 to 10 wherein R⁵ is C₁₋₁₂-alkyl, C₃₋₈-cycloalkyl, C₃₋₈-heterocycloalkyl, aryl or heteroaryl.

12. A compound according to claim 11 wherein R⁵ is C₁₋₆-alkyl, or C₃₋₈-cycloalkyl.

13. A compound according to claim 11 wherein R⁵ is heteroaryl, or aryl.

14. A compound according to any one of claims 1 to 13 wherein R⁶ is is C₁₋₁₂-alkyl, C₃₋₈-cycloalkyl, R⁸O, R⁸S, R⁸S(O), R⁸S(O)₂, (R⁹)(R¹⁰)N, R⁸C(Z)O, R⁸OC(Z)O, R⁸C(Z)N(R¹¹), R⁸OC(Z)N(R¹¹), R⁸S(O)ₙO, (R⁹)(R¹⁰)NC(Z)O, (R⁹)(R¹⁰)NS(O)₂O, R⁸S(O)₂N(R¹¹), or (R⁹)(R¹⁰)NS(O)₂N(R¹¹).

15. A compound according to claim 14 wherein R⁶ is C₁₋₆-alkyl, R⁸O, R⁸S, (R⁸)(R⁹)N, R⁸C(Z)O, R⁸C(Z)N(R¹¹), or R⁸S(O)₂N(R¹¹).

16. A compound according to claim 15 wherein R⁶ is is R⁸O, (R⁹)(R¹⁰)N, R⁸C(O)O, R⁸C(O)NH, or R⁸S(O)₂NH.

17. A compound according to any one of claims 1 to 16 wherein R⁷ is hydrogen, C₁₋₆-alkyl, C₂₋₄-alkenyl, or C₂₋₆-alkynyl.

18. A compound according to claim 17 wherein R⁷ is hydrogen.

19. A compound according to any one of claims 1 to 18 wherein R⁶ is R⁸O, (R⁹)(R¹⁰)N, R⁸C(O)O, R⁸C(O)NH, or R⁸S(O)₂NH and R⁷ is hydrogen.

20. A compound according to claim 19 wherein R⁶ is R⁸O or (R⁹)(R¹⁰)N and R⁷ is hydrogen.

21. A compound according to any one of claims 1 to 20 wherein R⁸, R⁹, R¹⁰, and R¹¹ are independently of each other hydrogen or C₁₋₆-alkyl, or R⁹ and R¹⁰ together with the nitrogen atom to which they are bound form a saturated heterocyclic ring having 5-6 ring members and which ring optionally contain 1 heteroatom, and which optionally is substituted by C₁₋₆-alkyl.

22. A compound according to any one of claims 1 to 21 wherein Y is hydroxy or C₁₋₆-alkoxy.

23. A compound according to claim 22 wherein Y is C₁₋₆-alkoxy.

24. A compound according to any one of claims 1 to 23 wherein n is 1 or 2.

25. A compound according to claim 24 wherein n is 1.

26. A compound according to any one of claims 1 to 25 wherein R¹ is COOH or heteroaryl; R² and R³ is independently of each other halogen or C₁₋₆-alkyl, or wherein both R² and R³ are halogen; R⁴ is C₁₋₁₂-alkyl, halogen, (R⁹)(R¹⁰)N, or R⁸C(Z)N(R¹¹); R⁵ is C₁₋₆-alkyl, C₃₋₈-cycloalkyl, aryl, or heteroaryl; R⁶ is C₁₋₆-alkyl, R⁸O, R⁸S, (R⁸)(R⁹)N, R⁸C(Z)O, R⁸C(Z)N(R¹¹), or R⁸S(O)₂N(R¹¹); R⁷ is hydrogen; R⁸, R⁹, R¹⁰ and R¹¹ are independently of each other hydrogen or C₁₋₆-alkyl, or R⁹ and R¹⁰ together with the nitrogen atom to which they are bound form a ring having 5-6 ring members and which ring optionally contain 1 heteroatom, and which optionally is substituted by C₁₋₆-alkyl; Y is hydroxy or C₁₋₆-alkoxy; and n is 1 or 2;
or pharmaceutically acceptable salts, stereoisomers or prodrugs thereof.

27. A compound according to any one of claims 1 to 26 wherein R¹ is COOH; R² and R³ is independently of each other halogen; R⁴ is C₁₋₁₂-alkyl; R⁵ is aryl or heteroaryl; R⁶ is R8O, (R⁸)(R⁹)N, R⁸C(O)O, R⁸C(O)NH, or R⁸S(O)₂NH; R⁷ is hydrogen; R⁸, R⁹, R¹⁰ and R¹¹ are independently of each other hydrogen or C₁₋₆-alkyl, or R⁹ and R¹⁰ together with the nitrogen atom to which they are bound form a ring having 5-6 ring members and which ring optionally contain 1 heteroatom, and which optionally is substituted by C₁₋₆-alkyl; Y is C₁₋₆-alkoxy; and n is 1;
or pharmaceutically acceptable salts, stereoisomers or prodrugs thereof.

28. A compound according to claim 1 said compound being:
3,5-Dibromo-4-[2-(1-hydroxyethyl)-5-isopropyl-4-methoxyphenoxy]phenylacetic acid (E1);
3,5-Dibromo-4-[2-(1-{3-indolyl}ethyl)-5-isopropyl-4-methoxyphenoxy]phenylacetic acid (E2);
4-[2-(2-Cyclopentyl-1-hydroxyethyl)-5-isopropyl-4-methoxyphenoxy]-3,5-dibromophenylacetic acid (E3);
3,5-Dibromo-4-{[2-(hydroxy(phenyl)methyl)]-5-isopropyl-4-methoxyphenoxy}phenylacetic acid (E4);
3,5-Dibromo-4-{5-isopropyl-4-methoxy-2-[(2-0{methylsulfonyl)ethoxy(phenyl)methyl]-phenoxy}phenylacetic acid (E5);
3,5-Dibromo-4-{2-[hydroxy(2-methylphenyl)methyl]-5-isopropyl-4-methoxyphenoxy}-phenylacetic acid (E6);
3,5-Dibromo-4-{2-[(2,4-difluorophenoxy)(2-methylphenyl)methyl]-5-isopropyl-4-methoxyphenoxy}phenylacetic acid (E7);
3,5-Dibromo-4-{2-[2-butylamino(2-methylphenyl)methyl]-5-isopropyl-4-methoxyphenoxy}phenylacetic acid (E8);
3,5-Dibromo-4-{2-[hydroxy(3-methylphenyl)methyl]-5-isopropyl-4-methoxy-phenoxy}-phenylacetic acid (E9);
3,5-Dibromo-4-[5-isopropyl-4-methoxy-2-(methoxy(3-methylphenyl)methyl)phenoxy]-phenylacetic acid (E10);
3,5-Dibromo-4-{2-[isopropoxy(3-methylphenyl)methyl]-5-isopropyl-4-methoxyphenoxy}-phenylacetic acid (E11);
4-{2-[Cyclohexyloxy(3-methylphenyl)methyl]-5-isopropyl-4-methoxyphenoxy}-3,5-dibrom ophenylacetic acid (E12);
3,5-Dibromo-4-{2-[(4-fluarophenoxy)(3-methylphenyl)methyl]-5-isopropyl-4-methoxyphenoxy}phenylacetic acid (E13);
3,5-Dibromo-4-{5-isopropyl-4-methoxy-2-[(4-methoxyphenoxy)(3-methylphenyl)-methyl]phenoxy}phenylacetic acid (E14);
3,5-Dibromo-4-{5-isopropyl-4-methoxy-2-[(3-methylphenyl)(4-nitrophenoxy)methyl]-phenoxy}phenylacetic acid (E15);
3,5-Dibromo-4-{2-[(4-aminophenoxy)(3-methylphenyl)methyl]-5-isopropyl-4-methoxyphenoxy}phenylacetic acid (E16);
3,5-Dibromo-4-{2-[(4-hydroxybenzoyloxy)(3-methylphenyl)methyl]-5-isopropyl-4-methox y]phenoxy}phenylacetic acid (E17);
4-{2-[Chloro(3-methylphenyl)methyl]-5-isopropyl-4-methoxyphenoxy}-3,5-dibromophenylacetic acid (E18);
4-{2-[Amino(3-methylphenyl)methyl]-5-isopropyl-4-methoxyphenoxy}-3,5-dibromophenylacetic acid (E19);
3,5-Dibromo-4-{2-[isopropylamino(3-methylphenyl)methyl]-5-isopropyl-4-methoxyphenoxy}phenylacetic acid (E20);
4-{2-[Cyclopropylamino(3-methylphenyl)methyl]-5-isopropyl-4-methoxyphenoxy}-3,5-dibromo-phenylacetic acid (E21);
3,5-Dibromo-4-{2-[(1-pyrrolidino)(3-methylphenyl)methyl]-5-isopropyl-4-methoxyphenoxy}phenylacetic acid (E22);
3,5-Dibromo-4-{2-[(1-piperidino)(3-methylphenyl)methyl]-5-isopropyl-4-methoxyphenoxy}phenylacetic acid (E23);
3,5-Dibromo-4-{2-[(2-methoxy-1-ethyl)amino(3-methylphenyl)methyl]-5-isopropyl-4-meth oxyphenoxy}phenylacetic acid (E24);
3,5-Dibromo-4-{2-[(2-{N,N-diethylamin}-1-ethyl)amino(3-methylphenyl)-methyl]-5-isopropyl-4-methoxyphenoxy}phenylacetic acid (E25);
3,5-Dibromo-4-{2-[(3-methylphenyl)(2-{1-piperidino}-1-ethyl)amino-methyl]-5-isopropyl-4-methoxyphenoxy}phenylacetic acid (E26);
3,5-Dibromo-4-{2-[(1-piperazino)(3-methylphenyl)methyl]-5-isopropyl-4-methoxyphenoxy}phenylacetic acid (E27);
3,5-Dibromo-4-{2-[4-methoxybenzylamino(3-methylphenyl)methyl]-5-isopropyl-4-methoxyphenoxy}phenylacetic acid (E28);
4-{2-[(3-Carboxyphenyl)amino(3-methylphenyl)methyl]-5-isopropyl-4-methoxyphenoxy}-3,5-dibromo-phenylacetic acid (E29);
3,5-Dibromo-4-{2-[(N-{4-hydroxybenzoyl}amino)(3-methylphenyl)methyl]-5-isopropyl-4-methoxyphenoxy}phenylacetic acid (E30);
3,5-Dibromo-4-{2-[(N-{4-methylbenzenesulfonyl}amino)(3-methylphenyl)-methyl]-5-isopropyl-4-methoxyphenoxy}phenylacetic acid (E31);
4-{2-[Benzylthio(3-methylphenyl)methyl]-5-isopropyl-4-methoxyphenoxy}3,5-dibromophenylacetic acid (E32);
3,5-Dibromo-4-{2-[(2-furylmethylthio)(3-methylphenyl)methyl]-5-isopropyl-4-methoxyphenoxy}phenylacetic acid (E33);
4-{2-[Carboxymethylthio(3-methylphenyl)methyl]-5-isopropyl-4-methoxyphenoxy}-3,5-dibromo-phenylacetic acid (E34);
3,5-Dibromo-4-{2-[(3-methylphenyl)phenylthio)methyl]-5-isopropyl-4-methoxyphenoxy}phenylacetic acid (E35);
3,5-Dibromo-4-{2-hydroxy(4-methylphenyl)methyl]-5-isopropyl-4-methoxyphenoxy}-phenylacetic acid (E36);
3,5-Dibromo-4-{2-[1-isopropoxy-1-(4-methylphenyl)methyl]-5-isopropyl-4-methoxyphenoxy}phenylacetic acid (E37);
3,5-Dibromo-4-{2-[(3-isopropylphenyl)hydroxymethyl]-5-isopropyl-4-methoxyphenoxy}-phenylacetic acid (E38);
3,5-Dibromo-4-{2-[(3-fluorophenyl)hydroxymethyl]-5-isopropyl-4-methoxyphenoxy}-phenylacetic acid (E39);
3,5-Dibromo-4-{2-[hydroxy(3-iodophenyl)methyl]-5-isopropyl-4-methoxyphenoxy}-phenylacetic acid (E40);
3,5-Dibromo-4-{2-[hydroxy(3-trifluorophenyl)methyl]-5-isopropyl-4-methoxyphenoxy}-phenylacetic acid (E41);
3,5-Dibromo-4-{5-isopropyl-4-methoxy-2-[methoxy-(3-trifluorophenyl)methyl]-phenoxy}phenylacetic acid (E42);
3,5-Dibromo-4-{2-[hydroxy(3-trifluoromethoxyphenyl)methyl]-5-isopropyl-4-methoxyphenoxy}phenylacetic acid (E43);
3,5-Dibromo-4-{2-[(3-dimethylaminophenyl)hydroxymethyl]-5-isopropyl-4-methoxyphenoxy}phenylacetic acid (E44);
3,5-Dibromo-4-{2-[(3,5-dimethylphenyl)hydroxymethyl]-5-isopropyl-4-methoxy-phenoxy}phenylacetic acid (E45);
4-{2-[Cyclohexyloxy(3,5-dimethylphenyl)methyl]-5-isopropyl-4-methoxyphenoxy}3,5-dibromophenylacetic acid (E46);
3,5-Dibromo-4-{2-[(3,5-difluorophenyl)hydroxymethyl]-5-isopropylmethoxyphenoxy}-phenylacetic acid (E47);
4-{2-(3-chloro-2-fluorophenyl)hydroxymethyl]-5-isopropyl-4-methoxyphenoxy}-3,5-dibromophenylacetic acid (E48);
4-{2-(3-Chloro-2-fluorophenyl)methoxymethyl]-5-isopropyl-4-methoxyphenoxy}3,5-dibromo-phenylacetic acid (E49);
3,5-Dibromo+[2-(4-fluoro-3-methylphenyl)hydroxymethyl)-5-isopropyl-4-methoxyphenoxy]phenylacetic acid (E50);
4-{5-(2-Cyclopentylethyl)-2-[hydroxy(3-methylphenyl)methyl]-4-methoxy-phenoxy}-3,5-di bromophenylacetic acid (E51);
4-{2-[hydroxy(3-methylphenyl)methyl]-5-iodo-4-methoxyphenoxy}-3,5-dibromophenylacetic acid (E52);
4-{5-acetamido-2-[hydroxy(3-methylphenyl)methyl]-4-methoxyphenoxy}-3,5-dibromophenylacetic acid (E53);
4-{2-[Hydroxy(3-methylphenyl)methyl]4-methoxy-5-(3-methylbenzamido)-phenoxy}-3,5-dibromophenylacetic acid (E54);
3,5-Dibromo-4-{4-hydroxy-2-[hydroxy(3-methylphenylvmethyl]-5 isopropylphenoxy}-phenylacetic acid (E55);
3,5-Dibromo-4-{2-[hydroxy(3-methylphenyl)methyl]4-isobutyloxy-5-isopropylphenoxy}-phenylacetic acid (E56);
3,5-Dibromo-4-{4-[2-fluoroethoxy]-2-[hydroxy-(3-methylphenyl)methyl]-5-isopropyl-phen oxy}phenylacetic acid (E57);
3,5-Dibromo-4-{2-[hydroxy(3-methylphenyl)methyl]-5 isopropyl-4-methoxyphenoxy}phenylpropionic acid (E58);
or pharmaceutically acceptable salts, stereoisomers or prodrugs thereof.

29. A compound according to any one of claims 1 to 28 for use in medical therapy.

30. A pharmaceutical composition comprising a compound according to any one of claims 1 to 28 together with a pharmaceutical diluent or carrier.

31. A process for making a pharmaceutical composition comprising combining a compound according to any one of claims 1 to 28 together with a pharmaceutical diluent or carrier.

32. A method for preventing, inhibiting, or treating a disease associated with a metabolism dysfunction in a mammal in need thereof, which comprises administering to a patient in need of treatment a therapeutically effective amount of a compound according to any one of claims 1 to 28.

33. A method for preventing, inhibiting, or treating a disease which is dependent on the expression of a glucocorticoid receptor regulated gene in a mammal in need thereof, which comprises administering to a patient in need of treatment a therapeutically effective amount of a compound according to any one of claims 1 to 28.

34. A method for eliciting a glucocorticoid receptor modulating effect in a mammal in need thereof, which comprises administering to a patient in need of treatment a therapeutically effective amount of a compound according to any one of claims 1 to 28.

35. A method according to claim 34, wherein the glucocorticoid receptor modulating effect is an antagonizing effect.

36. The method according to any one of claims 32 to 35 wherein the compound is a liver selective glucocorticoid receptor antagonist.

37. A method for preventing, inhibiting, or treating a disease in a mammal in need thereof, which comprises administering to a patient in need of treatment a therapeutically effective amount of a compound according to any one of claims 1 to 28, wherein the disease is selected from: Type 1 insulin dependent diabetes, Type 2 non-insulin dependent diabetes, Cushing's syndrome, inflammation, autoimmune disease, transplant rejection, neoplasm, leukemia, lymphoma, Cushings disease, adrenal disease, renal disease, cerebrovascular ischemia, hypercalcemia, cerebra edema, thrombocytopenia, inflammatory bowel disease, wound healing, HIV infection, central nervous system disease, spinal cord tumour, glaucoma, sleep disorder, depression, anxiety disorder, atherosclerosis, hypertension, osteoporosis, occular hypertension, nephrotoxicity, infarction, endometriosis, pregnancy disorder, psychosis, Alzheimers disease, cocaine use disorder, asthma, allergic rhinitis, conjuctivitis, rheumatoid arthritis, dermatitis, eczema, osteoarthritis, hypoglycemia, hyperinsulinemia, hyperlipidemia and obesity, or other endocrine disorders related to glucocorticoid hormones.

38. The method according to claim 37 wherein the disease is selected from Type 1 insulin dependent diabetes, Type 2 non-insulin dependent diabetes, Cushing's syndrome, and inflammation.

39. The use of a compound according to any one of claims 1 to 28 in the manufacture of a medicament for the therapeutic treatment or prevention of a disease or disorder, which is associated with a metabolism dysfunction.

40. The use of a compound according to any one of claims 1 to 28 in the manufacture of a medicament for the therapeutic treatment or prevention of a disease or disorder, which is dependent on the expression of a glucocorticoid receptor regulated gene.

41. The use of a compound according to any one of claims 1 to 28 in the manufacture of a medicament for the therapeutic treatment or prevention of a disease or disorder, which elicits a glucocorticoid receptor modulating effect in a mammal.

42. The use according to claim 41 wherein the glucocorticoid receptor modulating effect is an antagonizing effect.

43. The use according to any one of claims 39 to 42 wherein the compound is a liver selective glucocorticoid receptor antagonist.

44. The use of a compound according to any one of claims 1 to 28 in the manufacture of a medicament for the therapeutic treatment or prevention of a disease or disorder, wherein the disease or disorder is selected from: Type 1 insulin dependent diabetes, Type 2 non-insulin dependent diabetes, Cushing's syndrome, inflammation, autoimmune disease, transplant rejection, neoplasm, leukemia, lymphoma, Cushings disease, adrenal disease, renal disease, cerebrovascular ischemia, hypercalcemia, cerebra edema, thrombocytopenia, inflammatory bowel disease, wound healing, HIV infection, central nervous system disease, spinal cord tumour, glaucoma, sleep disorder, depression, anxiety disorder, atherosclerosis, hypertension, osteoporosis, occular hypertension, nephrotoxicity, infarction, endometriosis, pregnancy disorder, psychosis, Alzheimers disease, cocaine use disorder, asthma, allergic rhinitis, conjuctivitis, rheumatoid arthritis, dermatitis, eczema, osteoarthritis, hypoglycemia, hyperinsulinemia, hyperlipidemia and obesity, or other endocrine disorders related to glucocorticoid hormones.

45. The use according to claim 44 wherein the disease or disorder is selected from Type 1 insulin dependent diabetes, Type 2 non-insulin dependent diabetes, Cushing's syndrome, and inflammation.
